(19) United States
(12) Patent Application Publication
(10) Pub. No.: US 2019/0345562 A1
(43) Pub. Date: Nov. 14, 2019

(54) HOTSPOTS FOR CHROMOSOMAL REARRANGEMENT IN BREAST AND OVARIAN CANCERS

(71) Applicant: Genome Research Limited, Cambridge (GB)

(72) Inventors: Serena NIK-ZAINAL, Cambridge (GB); Dominik GLODZIK, Cambridge (GB)

(21) Appl. No.: 16/472,015

(22) PCT Filed: Dec. 22, 2017
(86) PCT No.: PCT/EP2017/084409

§ 371 Date: Jun. 20, 2019

(30) Foreign Application Priority Data
Dec. 22, 2016 (GB) 1621969.3

(51) Int. Cl. C12Q 1/6886; C12Q 1/6827; G16B 40/10; G16B 25/10; C12Q 1/6874

(52) U.S. Cl. CPC C12Q 1/6886; C12Q 1/6827; G16B 40/10; C12Q 2600/156; C12Q 1/6874; C12Q 2600/106; G16B 25/10

(57) ABSTRACT

The invention relates to the classification of breast and ovarian tumours, and in particular to the use of particular rearrangement signatures to identify tumours as deficient in homologous recombination repair (HR-deficient). The inventors have identified particular chromosomal “hotspots” of recombination in breast and ovarian cancers which permit the homologous recombination repair status of a cancer to be assessed by determining the presence of recombination events within those specific hotspots, rather than by analysing the entire cancer genome for the presence of rearrangement signatures as a whole.

### FIELD OF THE INVENTION

The invention relates to the classification of breast and ovarian tumours, and in particular to the use of particular rearrangement signatures to identify tumours as deficient in homologous recombination repair (HR-deficient).

### BACKGROUND TO THE INVENTION

Whole genome sequencing (WGS) has permitted unrestricted access to the human cancer genome, triggering the hunt for driver mutations that could confer selective advantage in all parts of human DNA. Recurrent somatic mutations in coding sequences are often interpreted as driver mutations particularly when supported by transcriptomic changes or functional evidence. However, recurrent somatic mutations in non-coding sequences are less straightforward to interpret. Although TERT promoter mutations in malignant melanoma^{2,3} and NOTCH1 3′ region mutations in chronic lymphocytic leukaemia⁴ have been successfully demonstrated as driver mutations, multiple non-coding loci have been highlighted as recurrently mutated but evidence supporting these as true drivers remains lacking. Indeed, in a recent exploration of 560 breast cancer whole genomes¹, the largest cohort of WGS cancers to date, statistically significant recurrently mutated non-coding sites (by substitutions and insertions/deletions (indels)) were identified but alternative explanations for localized elevation in mutability such as a propensity to form secondary DNA structures were observed¹.

These efforts have been focused on recurrent substitutions and indels and an exercise seeking sites that are recurrently mutated through rearrangements has not been formally performed. Such sites could be indicative of driver loci under selective pressure (such as amplifications of ERBB2 and CCND1) or could represent highly mutable sites that are simply prone to double-strand break (DSB) damage. Sites that are under selective pressure generally have a high incidence in a particular tissue-type, are highly complex and comprise multiple classes of rearrangement including deletions, inversions, tandem duplications and translocations. By contrast, sites that are simply breakable may show a low frequency of occurrence and demonstrate a preponderance of a particular class of rearrangement, a harbinger of susceptibility to a specific mutational process.

### SUMMARY OF THE INVENTION

The inventors have previously found that subsets of certain cancers are characterised by particular “rearrangement signatures” which indicate a likely failure of DNA double strand repair by homologous recombination. Knowing the homologous recombination repair status of a cancer may inform decisions on treatment, since some agents are more effective against cancers with deficiency in homologous recombination repair, commonly referred to as “HR-deficient” cancers, than against other cancers.

The inventors have now identified particular chromosomal “hotspots” of recombination in breast and ovarian cancers. Thus it may be possible to gauge the homologous recombination repair status of a cancer by determining the presence of recombination events within those specific hotspots, rather than by analysing the entire cancer genome for the presence of rearrangement signatures as a whole.

The invention provides a method of classifying a breast cancer, comprising

testing DNA from said breast cancer for the presence of chromosomal rearrangement within 10 or more of the rearrangement hotspots defined in Table 1; and
classifying said breast cancer as HR-deficient if rearrangement is identified in at least one of said rearrangement hotspots.

Typically, the method will comprise testing for the presence of chromosomal rearrangement within 15 or more, within 20 or more, within 25 or more, within 26 or more, 27 or more, 28 or more, 29 or more, 30 or more, 31 or more, 32 or more, or all 33 of the hotspots defined in Table 1.

The confidence of correctly classifying the cancer as HR-deficient increases with the number of hotspots in which chromosomal rearrangement is identified. Thus in some embodiments the cancer may be classified as HR-deficient only if rearrangement is identified in each of a plurality of hotspots, e.g. in each of at least 2 hotspots, at least 3 hotspots, at least 4 hotspots, at least 5 hotspots, at least 6 hotspots, at least 7 hotspots, at least 8 hotspots, at least 9 hotspots, at least 10 hotspots, or even more. It is presently believed that a high level of confidence is provided by identification of chromosomal rearrangement in each of at least 3 hotspots, increasing with identification of rearrangement in at least 4 hotspots or at least 5 hotspots, with a confidence approaching 100% for identification of rearrangement in each of at least 6 hotspots.

The invention further provides a method of determining a therapy for a subject having breast cancer, the method comprising

testing DNA from said breast cancer for the presence of chromosomal rearrangement within 10 or more of the rearrangement hotspots defined in Table 1; and
selecting the subject for treatment with an agent for treatment of HR-deficient cancers if rearrangement is identified in at least one of said rearrangement hotspots.

It may be desirable to select the subject for treatment with the relevant agent only if chromosomal rearrangement is identified in each of at least 2 hotspots, at least 3 hotspots, at least 4 hotspots, at least 5 hotspots, at least 6 hotspots, at least 7 hotspots, at least 8 hotspots, at least 9 hotspots, at least 10 hotspots, or even more; e.g. in each of at least 3 hotspots, at least 4 hotspots, at least 5 hotspots or at least 6 hotspots.

The method may comprise the step of classifying the cancer as HR-deficient. Thus the invention further provides a method of determining a therapy for a subject having a breast cancer comprising performing a method of classification as described herein and selecting said subject for treatment with an agent for treatment of HR-deficient cancers if said cancer is classified as HR-deficient.

The method may comprise the step of treating the subject with said agent.

The invention further provides an agent for treatment of HR-deficient cancers, for use in the treatment of breast cancer in a subject (i) selected by a method as described herein, or (ii) having a breast cancer which has been determined to be HR-deficient by a method as described herein.

The invention further provides the use of an agent for treatment of HR-deficient cancers in the preparation of a medicament for the treatment of breast cancer, wherein the medicament is for administration to a subject (i) selected by a method as described herein, or (ii) having a breast cancer which has been determined to be HR-deficient by a method as described herein.

The invention further provides a method of treatment of breast cancer, in a subject (i) selected by a method as described herein, or (ii) having a breast cancer which has been determined to be HR-deficient by a method as described herein, the method comprising administering an agent for treatment of HR-deficient cancers to the subject.

The hotspot designated B23 (peak_RS1_chr6_151.8mb) encompasses the estrogen receptor 1 (ESR1) gene. Samples containing tandem-duplicated ESR1 have high expression levels of ESR1, similar to those of so-called “ER positive” cancers, even when just a single tandem duplication is present. This is surprising, since cancers which are ER-positive as a result of gene amplification (rather than other mutations) are conventionally expected to have a considerably copy number, e.g. of around 10 copies, or even more.

Thus a cancer having a rearrangement, especially a tandem duplication, within hotspot B23 may have increased copy number and/or expression of ESR1, and so may be suitable for treatment with an agent for treatment of estrogen receptor positive (“ER-positive”) cancers. A finding of rearrangement within this hotspot may therefore enable a cancer to be designated “ER-positive”.

Analysis of ER receptor status may be performed in conjunction with an analysis of HR-deficiency, or independently.

Thus the invention provides a method of classifying a breast cancer, comprising testing DNA from said breast cancer for the presence of chromosomal rearrangement within hotspot B23 (peak_RS1_chr6_151.8mb) defined in Table 1; and classifying said breast cancer as ER-positive if rearrangement is identified in said hotspot.

The invention further provides a method of determining a therapy for a subject having breast cancer, the method comprising

testing DNA from said breast cancer for the presence of chromosomal rearrangement within hotspot B23 (peak_RS1_chr6_151.8mb) defined in Table 1; and
selecting the subject for treatment with an agent for treatment of ER-positive cancers if rearrangement is identified in said hotspot.

The method may comprise the step of classifying the cancer as ER-positive. Thus the invention further provides a method of determining a therapy for a subject having a breast cancer comprising performing a method of classification as described herein and selecting said subject for treatment with an agent for treatment of ER-positive cancers if said cancer is classified as ER-positive.

The method may comprise the step of treating the subject with said agent.

The invention further provides an agent for use in the treatment of ER-positive cancers, for use in the treatment of breast cancer in a subject (i) having a breast cancer which has been determined to be ER-positive by a method as described herein, or (ii) selected by a method as described herein.

The invention further provides the use of an agent for treatment of ER-positive cancers in the preparation of a medicament for the treatment of breast cancer, wherein the medicament is for administration to a subject (i) having a breast cancer which has been determined to be ER-positive by a method as described herein, or (ii) selected by a method as described herein.

The invention further provides a method of treatment of breast cancer, in a subject (i) having a breast cancer which has been determined to be ER-positive by a method as described herein, or (ii) selected by a method as described herein, the method comprising administering an agent for treatment of ER-positive cancers to the subject.

Any of the methods described may comprise an additional step of testing the copy number of the ESR1 gene, and/or testing the ER status of the cancer, in order to confirm the classification and eliminate any false-positive identification. This may involve testing for expression of ESR1 receptor protein or mRNA. The test may be qualitative (determining whether or not ESR1 is expressed) or quantitative (determining level of expression). The expression level determined may be compared, for example, to previously-determined reference values or to normal breast tissue from the subject.

The invention further provides a method of classifying an ovarian cancer, comprising testing DNA from said ovarian cancer for the presence of chromosomal rearrangement within 2 or more of the rearrangement hotspots defined in Table 5; and classifying said ovarian cancer as HR-deficient if rearrangement is identified in at least one of said rearrangement hotspots.

Typically, the method will comprise testing for the presence of chromosomal rearrangement within 3 or more, within 4 or more, within 5 or more, within 6 or more, or within all 7 hotspots defined in Table 5.

The confidence of correctly classifying the cancer as HR-deficient increases with the number of hotspots in which chromosomal rearrangement is identified. Thus in some embodiments the cancer may be classified as HR-deficient only if chromosomal rearrangement is identified in each of at least 2 hotspots, at least 3 hotspots, at least 4 hotspots, at least 5 hotspots, at least 6 hotspots, or all 7 hotspots.

The invention further provides a method of determining a therapy for a subject having an ovarian cancer, the method comprising

testing DNA from said ovarian cancer for the presence of chromosomal rearrangement within 2 or more of the rearrangement hotspots defined in Table 5; and
selecting the subject for treatment with an agent for treatment of HR-deficient cancers if rearrangement is identified in at least one of said rearrangement hotspots.

It may be desirable to select the subject for treatment with the relevant agent only if chromosomal rearrangement is identified in each of at least 2 hotspots, at least 3 hotspots, at least 4 hotspots, at least 5 hotspots, at least 6 hotspots, or all 7 hotspots.

The method may comprise the step of classifying the cancer as HR-deficient. Thus the invention further provides a method of determining a therapy for a subject having an ovarian cancer comprising performing a method of classification as described herein and selecting said subject for treatment with an agent for treatment of HR-deficient cancers if said cancer is classified as HR-deficient.

The method may comprise the step of treating the subject with said agent.

The invention further provides an agent for treatment of HR-deficient cancers, for use in the treatment of ovarian cancer in a subject (i) selected by a method as described herein, or (ii) having an ovarian cancer which has been determined to be HR-deficient by a method as described herein.

The invention further provides the use of an agent for treatment of HR-deficient cancers in the preparation of a medicament for the treatment of ovarian cancer, wherein the medicament is for administration to a subject (i) selected by a method as described herein, or (ii) having an ovarian cancer which has been determined to be HR-deficient by a method as described herein.

The invention further provides a method of treatment of ovarian cancer, in a subject (i) selected by a method as described herein, or (ii) having an ovarian cancer which has been determined to be HR-deficient by a method as described herein, the method comprising administering an agent for treatment of HR-deficient cancers to the subject.

The presence or absence of chromosomal rearrangement in each tested hotspot is typically determined by comparison with one or more reference sequence(s) for the same hotspot.

Thus the method may comprise determining a data set for each of the tested hotspots from the cancer DNA and comparing each data set from the cancer DNA with a corresponding reference data set to identify any chromosomal rearrangements within each tested hotspot in the cancer DNA.

The term “reference sequence” is used here to refer to a specific single sequence used for comparison with a sequence from a cancer sample in order to identify instances of rearrangement in the cancer genome. The term “reference data set” may be used to refer to data derived from one or more reference sequences in any given hotspot. The term “reference genome” is used to refer to a genome comprising any given reference sequence, and may be used to refer to a collection of reference sequences.

Thus each data set from the cancer DNA is compared with a corresponding reference data set derived from the reference sequence or reference genome in order to detect the presence (and optionally type and/or frequency) of rearrangement in the cancer DNA. The content of each data set will depend on the precise format of the particular experiment and the methodology used, but may include full sequence data, absolute or relative positions of particular loci or pairs of loci. etc.

The reference genome(s), sequence(s) and data set(s) derived therefrom are typically representative of normal (i.e. healthy, non-neoplastic) tissue and may be obtained from any suitable source, including publicly-available or proprietary databases of representative genomic DNA sequences. The reference sequence or genome may be from a single individual, or a compilation or consensus representative of a particular population. The reference genome(s) or sequence(s) may be pre-determined, or may be determined as part of the method of the invention, alongside the cancer sample. However, it is generally preferred that the reference genome or sequence is derived using DNA (“reference DNA”) from healthy tissue (“reference tissue”) from the same subject, to ensure that any chromosomal rearrangement(s) identified in the cancer is specifically associated with the process of neoplasia and is not a feature of the subject's “normal” genome.

The methods may be performed on genomic DNA.

Thus the methods may comprise providing a sample containing genomic DNA from the cancer. For example, the sample may comprise one or more cells from the cancer (e.g. from peripheral blood or from a biopsy of the cancer) or may simply contain free genomic DNA (e.g. circulating tumour DNA from peripheral blood).

The methods may independently comprise providing a sample containing reference genomic DNA, e.g. a sample containing normal reference tissue, e.g. from the same individual.

In either case, the method may comprise isolating genomic DNA from any samples provided, whether from the cancer or the reference tissue. Whether or not any isolation takes place, the method may comprise further steps of preparing the genomic DNA for analysis. Such preparation steps will depend on the chosen method of analysis and may include fragmentation (by physical or enzymatic means), fractionation, amplification (typically by enzymatic means), enrichment for specific sequences or regions (e.g. hotspots), linkage to adapters, etc.

For example, the method may involve a step of enriching a sample for hotspot sequences.

The method may comprise contacting a sample of fragmented genomic DNA from the subject with a hybridisation probe capable of hybridising specifically with a sequence from one of the hotspots to be tested. The method may comprise the further step of isolating the hybridising genomic DNA. Thus, it is possible to enrich a sample for sequences within hotspot regions, thus enabling the subsequent sequencing to be targeted only to the hotspots and not to the entire genome.

The method may employ a plurality of hybridisation probes, wherein each said probe is capable of hybridising specifically to a sequence from one of said hotspots. Typically, at least one probe is provided with specificity for each hotspot to be tested. Multiple probes may be provided for each hotspot to be tested.

Each probe may be provided on a solid support, such as a micro-array or a bead. A single support may carry a single probe or a plurality of probes. For example, a micro-array may carry a plurality of different probes, each having a defined spatial location on the array. A bead may carry multiple copies of the same probe or a plurality of probes of different sequences.

It may not be necessary in all cases to determine a full sequence of a hotspot in order to identify the presence (or absence) of chromosomal rearrangement (although this may provide the most reliable results, maximising the chance of identifying all informative rearrangements while minimising false positive results). It may be sufficient to determine a sequence (full or partial) of a portion of a hotspot, determine a change in copy number of a particular sequence within a hotspot, or to determine whether a change in distance (chromosomal length) has taken place between two specific loci within the hotspot in the cancer DNA as compared to the reference.

Analysis of the DNA from the cancer and, where appropriate, the reference DNA, may be carried out by any suitable method capable of detecting chromosomal rearrangement events, including sequencing and hybridisation methodologies.

Suitable sequencing techniques include paired end sequencing (or mate-pair sequencing), targeted sequencing, single molecule real-time sequencing, ion semiconductor (Ion Torrent) sequencing, sequencing by synthesis, sequencing by ligation (SOLiD), nano-pore sequencing and pyrosequencing, as well as more traditional techniques of cloning followed by chain termination (Sanger) sequencing.

Hybridisation-based techniques typically employ microarrays and may involve comparative hybridisation to compare reference and cancer sequences. Suitable techniques include array comparative genomic hybridisation (array CGH).

The subject is typically human, but may be any mammal. For example, the subject may be a primate (e.g. ape, Old World monkey, New World monkey), rodent (e.g. mouse or rat), canine (e.g. domestic dog), feline (e.g. domestic cat), equine (e.g. horse), bovine (e.g. cow), caprine (e.g. goat), ovine (e.g. sheep) or lagomorph (e.g. rabbit). It will be apparent that the subject is generally a female of the relevant species.

### Brief Description of the Tables

Table 1: Hotspots of rearrangement signatures RS1 identified through PCF-based method.
Table 2: Hotspots of rearrangement signature RS3 identified through PCF-based method.
Table 3. Genomic features of the RS1 hotspots. Comparison with the rest of tandem-duplicated genome with respect to: breast cancer susceptibility SNPs, breast tissue super-enhancers, non-breast super-enhancers, known oncogenes, promoters, enhancers, broad fragile sites, narrow fragile sites. A, Description of headers. B, Associations.
Table 4: Modelling the effects of RS1 tandem duplications on gene expression. Rows—coefficients used in the regression models. Columns—experiments with different sets of genes. In the table we show the fitted values of regression coefficients.
Table 5: Hotspots of rearrangement signatures RS1 identified through PCF-based method in ovarian tumours.

### DETAILED DESCRIPTION OF THE INVENTION

Somatic rearrangements contribute to the mutagenized landscape of human cancer genomes. The present inventors systematically interrogated catalogues of somatic rearrangements of 560 breast cancers¹ to identify hotspots of recurrent rearrangements, specifically tandem duplications, because of previous anecdotal reports of tandem duplications that recurred in different patients.

In all, 77,695 rearrangements including 59,900 intra-chromosomal (17,564 deletions, 18,463 inversions and 23,873 tandem duplications) and 17,795 inter-chromosomal translocations were identified in this cohort previously. The distribution of rearrangements within each cancer was complex; some had few rearrangements without distinctive patterns, some had collections of focally occurring rearrangements such as amplifications, whereas many had rearrangements distributed throughout the genome—indicative of very different set of underpinning mutational processes.

Thus, large, focal collections of “clustered” rearrangements were first separated from rearrangements that were widely distributed or “dispersed” in each cancer, then distinguished by class (inversion, deletion, tandem duplication or translocation) and size (1-10 kb, 10-100 kb, 100 kb-1 Mb, 1-10 Mb, more than 10 Mb)¹, before a mathematical method for extracting mutational signatures was applied⁵. Six rearrangement signatures were extracted (RS1-RS6) representing discrete rearrangement mutational processes in breast cancer¹.

Two distinctive mutational processes in particular were associated with dispersed tandem duplications. RS1 and RS3 are mostly characterized by large (>100 kb) and small (<10 kb) tandem duplications, respectively. Although both are associated with tumors that are deficient in homologous recombination (HR) repair⁶⁻⁹, RS3 is specifically associated with inactivation of BRCA1. Thus, the two types of signature appear to represent distinct biological defects.

A set of 33 hotspots has been identified, dominated by the RS1 mutational process, and characterized by long (>100 kb) tandem duplications¹. Intuitively, a hotspot of mutagenesis that is enriched for a particular mutational signature implies a propensity to DNA double-strand break (DSB) damage and specific recombination-based repair mutational mechanisms that could explain these tandem duplication hotspots.

Whether these RS1-enriched hotspots are purely scars of mutational processes or are selected for, we postulate that these 33 loci could be used as potential biomarkers for positively identifying HR-deficient tumors.

In particular, we find that having a large number of RS1-enriched hotspots is predictive of HR-deficiency, specifically, identifying tandem duplication-enriched BRCA1-null or BRCA1-intact tumors. Previously, we identified breast cancer samples in the cohort of 560 patients as being HR-deficient based on mutation patterns derived from substitutions, indels and rearrangements²—HR-deficient tumors could be classified into tandem duplication-enriched BRCA1-null or BRCA1-intact groups, while BRCA2-null tumors were mainly characterized by large-scale deletions. In the present analysis, it was found that 67% of samples with rearrangements at 2 or more hotspots were HR-deficient, 82% of samples with rearrangements at 3 or more hotspots or 4 or more hotspots were HR-deficient.

Furthermore, 89% of samples with 5 or more hotspots and 100% of samples with 6 or more hotspots were HR-deficient. Thus, these loci of RS1-enriched hotspots are capable of serving as markers of defective HR repair. The panel of 33 loci does not have the sensitivity to detect all tumors with defective HR repair. However, having a number of mutated loci (four to six) in a tumor has strong positive predictive value for HR deficiency, with important clinical implications.

Cohorts of 96 pancreatic cancers and 73 ovarian cancers were also analysed. While no RS1-enriched hotspots were identified in the pancreatic cancers, a set of 7 RS1-enriched hotspots was identified in the ovarian cancers.

### Classification of Breast Cancers

The 33 hotspots which characterise breast cancers are defined by the coordinates provided in Table 1. All coordinates correspond to the Genome Reference Consortium Human genome build 37 (GRCh37) patch release 13 (GRCh37.p13), dated 28 Jun. 2013.

A method of classifying a breast tumour comprises testing for the presence of chromosomal rearrangement within 10 or more of the RS1 rearrangement hotspots defined in Table 1, e.g. within 15 or more, within 20 or more, within 25 or more, within 26 or more, within 27 or more, within 28 or more, within 29 or more, within 30 or more, within 31 or more, within 32, or within all 33 of the hotspots defined in Table 1.

A set of 32 hotspots may omit any one of the hotspots listed in Table 1, e.g. B1, B2, B3, B4, B5, B6, 87, B8, B9, B10, B11, B12, B13, B14, B15, B16, B17, B18, B19, 8B20, 8B21, B22, B23, B24, B25, B26, B27, B28, 8B29, B30, B31, 8B32 or B33.

A set of 31 hotspots may additionally omit any other hotspot listed in Table 1, and so on for smaller sets of hotspots.

For example, a set of 31 hotspots may omit any of the following hotspots:

### B1 and any one of B2, B3, B4, B5, B6, B7, B8, B9, 810, B11, B12, B13, B14, B15, 816, B17, 8B18, B19, B20, B21, B22, B23, B24, B25, B26, B27, B28, B29, B30, B31, B32 or B33;

### B2 and any one of B1, B3, B4, B5, B6, B7, B8, B9, 810, 811, 812, 813, B14, B15, 816, B17, 8B18, 8B19, 8B20, 8B21, 8B22, 8B23, 8B24, 8B25, 8B26, 8B27, B28, B29, 8B30, 8B31, 832 or B33;

### B3 and any one of 81, B2, B4, B5, B6, B7, B8, B9, B10, B11, B12, 813, B14, 815, 816, B17, 8B18, 8B19, 8B20, 8B21, 8B22, 8B23, 8B24, 8B25, 8B26, 8B27, 8B28, 8B29, 8B30, B31, 8B32 or 833;

### B4 and any one of B1, B2, B3, B5, B6, B7, B8, B9, B10, B11, B12, 813, B14, B15, 816, B17, 8B18, 8B19, 8B20, 8B21, 8B22, 8B23, 8B24, 8B25, B26, B27, B28, B29, B30, 8B31, 832 or B33;

### B5 and any one of 81, B2, B3, B4, B6, B7, B8, B9, 810, B11, B12, 813, 814, 815, 816, B17, 8B18, 8B19, 8B20, 8B21, 8B22, 8B23, 8B24, 8B25, 8B26, 8B27, 8B28, 8B29, 8B30, 8B31, 832 or 833;

B6 and anyone of B1, B2, B3, B4, 85, B7, B8, B9, B10, 8B11, B12, 8B13, 8B14, 8B15, B16, B17, B18, 8B19, B20, 8B21, B22, 8B23, B24, B25, B26, 8B27, B28, 8B29, B30, B31, B32 or 8B33;

### B7 and any one of B1, B2, 83, B4, B5, B6, B8, B9, B10, B11, B12, 8B13, 8B14, B15, B16, B17, 8B18, B19, 8B20, 8B21, 8B22, B23, B24, B25, 8B26, B27, B28, B29, B30, B31, B32 or B33;

B8 and anyone of B1, B2, B3, B4, B5, B6, B7, B9, B10, B11, B12, B13, B14, B15, B16, B17, B18, 8B19, B20, B21, B22, B23, B24, B25, 8B26, B27, B28, B29, 8B30, 8B31, 8B32 or B33;

### B9 and any one of B1, B2, B3, B4, B5, B6, B7, B8, B10, B11, B12, B13, 8B14, B15, 8B16, B17, B18, B19, 8B20, 8B21, 8B22, B23, 8B24, 8B25, 8B26, 8B27, 8B28, 8B29, 8B30, 8B31, 8B32 or B33;

### B10 and any one of 81, B2, B3, B4, B5, B6, B7, B8, B9, B11, B12, 8B13, B14, 8B15, 8B16, B17, 8B18, 8B19, 8B20, B21, 8B22, 8B23, 8B24, 8B25, 8B26, B27, B28, B29, 8B30, 8B31, 8B32 or 833;

B11 and anyone of B1, B2, 83, B4, 85, B6, 87, B8, 89, B10, 8B12, 8B13, 8B14, 8B15, B16, B17, 8B18, 8B19, 8B20, 8B21, 8B22, 8B23, 8B24, 8B25, 8B26, 8B27, B28, B29, 8B30, B31, 8B32 or 8B33;

### B12 and any one of 81, B2, 83, 84, 85, 86, B7, 88, B9, B10, B11, 8B13, 8B14, 815, 8B16, B17, 8B18, 8B19, B20, B21, B22, B23, B24, 8B25, 8B26, 8B27, 8B28, 8B29, B30, 8B31, 8B32 or 8B33;

### B13 and any one of B1B2, B3, B4, B5, B6, B7, B8, B9, B10, B11, B12, B14, B15, B16, B17, 8B18, B19, 820, 8B21, 8B22, 8B23, B24, 8B25, 8B26, 8B27, 8B28, 8B29, 8B30, 8B31, 8B32 or 833;

### B14 and any one of B1, B2, B3, B4, B5, B6, B7, B8, B9, B10, B11, B12, B13, B15, B16, B17, 8B18, 8B19, 8B20, 8B21, 8B22, 8B23, 8B24, 8B25, 8B26, B27, 828, B29, 8B30, 8B31, 8B32 or 833;

B15 and anyone of 81, B2, 83, 84, B5, B6, 87, B8, B9, B10, B11, B12, B13, 8B14, 8B16, B17, 818, B19, 8B20, 8B21, 8B22, 8B23, 8B24, 8B25, 8B26, 8B27, 8B28, 8B29, 830, 8B31, 8B32 or 8B33;
B16 and anyone of 81, 82, B3, 84, 85, B6, 87, B8, B9, B10, B11, 8B12, 8B13, 8B14, 8B15, B17, B18, 8B19, B20, 8B21, 8B22, 8B23, 8B24, 8B25, 8B26, 8B27, 8B28, 8B29, 8B30, 8B31, B32 or 8B33;

### B17 and any one of B1, B2, 83, 84, 85, B6, 87, 88, B9, B10, B11, B12, B13, 8B14, 8B15, B16, 8B18, 8B19, B20, 8B21, B22, B23, 8B24, 8B25, 8B26, 8B27, 8B28, 8B29, 8B30, 8B31, 8B32 or 833;

### B18 and any one of B1, B2, 83, 84, B5, 86, 87, B8, 89, B10, B11, 8B12, 8B13, 8B14, 8B15, B16, 8B17, B19, B20, B21, B22, B23, 8B24, 8B25, 8B26, 8B27, 8B28, 8B29, 8B30, 8B31, 8B32 or B33;

### B19 and any one of B1, B2, 83, 4, B5, B6, B7, B8, 89, B10, B11, B12, 8B13, 8B14, 8B15, 816, 8B17, 8B18, 8B20, B21, B22, B23, 8B24, 8B25, 8B26, 8B27, 8B28, 8B29, 8B30, 8B31, 8B32 or 833;

B20 and anyone of B1, B2, B3, B4, B5, 86, B7, 88, B9, B10, B11, B12, 8B13, 8B14, 8B15, B16, 8B17, B18, 8B19, 8B21, 8B22, B23, B24, B25, B26, 8B27, 8B28, 8B29, 8B30, 8B31, B32 or 8B33;

### B21 and any one of B1, B2, B3, 84, 85, 86, B7, 88, B9, B10, 811, B12, 8B13, 8B14, 8B15, B16, B17, 8B18, 819, 8B20, B22, B23, 8B24, B25, B26, B27, B28, B29, 8B30, B31, B32 or B33;

### B22 and any one of B1, B2, B3, B4, B5, B6, B7, B8, B9, B10, B11, B12, B13, B14, B15, B16, B17, 8B18, 8B19, B20, 8B21, B23, B24, B25, B26, 8B27, 8B28, 8B29, 8B30, B31, B32 or B33;

### B23 and any one of B1, B2, B3, B4, 85, B6, B7, B8, B9, B10, B11, B12, B13, 814, B15, B16, 8B17, 8B18, 8B19, 8B20, 821, B22, 8B24, 8B25, 8B26, 8B27, 8B28, 8B29, 8B30, 8B31, 8B32 or 833;

### B24 and any one of 81, B2, 83, 84, B5, 86, 87, 88, 89, B10, B11, B12, B13, B14, B15, B16, 8B17, 8B18, 8B19, 8B20, 8B21, B22, 8B23, 8B25, 826, 8B27, 8B28, 8B29, 8B30, 8B31, 8B32 or 833;

### B25 and any one of B1, 82, B3, 84, 85, B6, 87, B8, 89, B10, 811, B12, 8B13, 8B14, 8B15, B16, 8B17, 8B18, 8B19, 8B20, 8B21, B22, 8B23, 8B24, 8B26, 8B27, 8B28, 8B29, 8B30, 8B31, 8B32 or 8B33;

### B26 and any one of B1, 82, 83, 84, B5, 86, 87, B8, 89, B10, 811, B12, 813, 8B14, 8B15, B16, 8B17, 8B18, 8B19, 8B20, 8B21, B22, 8B23, 8B24, 8B25, 8B27, 828, 8B29, 8B30, 8B31, 8B32 or 8B33;

### B27 and any one of B1, B2, B3, B4, B5, B6, B7, B8, B9, B10, B11, B12, B13, B14, B15, B16, 8B17, 8B18, 8B19, 8B20, 8B21, B22, 8B23, 8B24, 8B25, 8B26, 8B28, 8B29, 8B30, 8B31, 832 or 833;

### B28 and any one of B1, 82, 83, 84, B5, B6, 87, 88, B9, 8B10, B11, 812, B13, B14, B15, B16, 8B17, 8B18, 8B19, 8B20, 8B21, B22, 8B23, 8B24, 8B25, 8B26, 8B27, 8B29, 8B30, 8B31, 8B32 or 8B33;

B29 and anyone of 81, B2, 83, 84, B5, 86, 87, 88, B9, B10, B11, B12, B13, 8B14, 8B15, B16, 8B17, 8B18, 8B19, 8B20, 821, 822, 823, 8B24, 8B25, 8B26, 8B27, 8B28, 8B30, 8B31, 8B32 or 833;

### B30 and any one of 81, B2, 83, B4, 85, 86, B7, 88, B9, B10, 8B11, B12, 8B13, 8B14, 8B15, B16, 8B17, 8B18, 8B19, 8B20, 8B21, B22, 823, 8B24, 8B25, 8B26, 8B27, 8B28, 8B29, 8B31, 8B32 or 833;

### B31 and any one of B1, 82, B3, 84, B5, 86, B7, 88, B9, B10, B11, B12, B13, B14, B15, B16, 8B17, 8B18, 8B19, 8B20, 8B21, B22, 8B23, 8B24, 8B25, 8B26, 8B27, 8B28, 8B29, 8B30, 8B32 or 8B33;

### B32 and any one of 81, B2, 83, B4, 85, 86, B7, 88, 89, B10, 811, B12, 8B13, 8B14, 8B15, B16, 8B17, 8B18, B19, 8B20, 8B21, B22, 8B23, 8B24, B25, 8B26, 8B27, 8B28, 8B29, 8B30, 8B31 or 833;

B33 and anyone of B1, 82, 83, B4, 85, B6, 87, B8, 89, B10, B11, B12, B13, 8B14, 8B15, B16, 8B17, 8B18, 8B19, 8B20, 8B21, B22, 8B23, 8B24, 8B25, 8B26, 827, 8B28, 8B29, 8B30, 8B31 or 8B32.

A cancer may be classified as HR-deficient if it has at least one rearrangement within any of the hotspots tested. However, the confidence of correctly classifying the cancer as HR-deficient increases with the number of hotspots in which chromosomal rearrangement is identified. Thus, in some embodiments, the cancer may be classified as HR-deficient only if rearrangement is identified in each of a plurality of hotspots, e.g. in each of at least 2 hotspots, at least 3 hotspots, at least 4 hotspots, at least 5 hotspots, at least 6 hotspots, at least 7 hotspots, at least 8 hotspots, at least 9 hotspots, at least 10 hotspots, or even more.

It is presently believed that a high level of confidence is provided by identification of chromosomal rearrangement in each of at least 3 hotspots, increasing with identification of rearrangement in at least 4 hotspots or at least 5 hotspots, with a confidence approaching 100% for identification or rearrangement in each of at least 6 hotspots.

A breast cancer which displays rearrangement, particularly a tandem duplication, in the hotspot containing ESR1 (B23) may have elevated levels of estrogen receptor expression and may be suitable for therapy with agents for treatment of ER-positive cancers. A finding of rearrangement, particularly duplication, in this hotspot may therefore enable a cancer to be designated as ER-positive, and selected for therapy with an agent for treatment of ER-positive cancer.

Any of the methods of the invention, insofar as they relate to this hotspot, may therefore comprise an additional step of testing the copy number of the ESR1 gene, to confirm that the ESR1 gene is indeed duplicated and that any duplication does not simply affect another region of that hotspot. The cancer may be designated as ER-positive, or selected for therapy with an agent for treatment of ER-positive cancers, if the copy number has increased (i.e. if an individual chromosome has two or more copies of the gene, or if the cancer genome as a whole has three or more copies of the gene.)

Additionally or alternatively, the method may include a step of testing the ER status of the cancer, in order to confirm the classification and eliminate any false-positive identification. This may involve testing for expression of ESR1 receptor protein or mRNA. The test may be qualitative (i.e. determining whether or not ESR1 mRNA or protein is expressed) or quantitative (i.e. determining the level of expression of ESR1 mRNA or protein). The expression level determined may be compared, for example, to previously-determined reference values or to normal breast tissue from the subject.

### Classification of Ovarian Cancers

The 7 hotspots which characterise ovarian cancers are defined by the coordinates provided in Table 5. All coordinates correspond to the Genome Reference Consortium Human genome build 37 (GRCh37) patch release 13 (GRCh37.p13), dated 28 Jun. 2013.

A method of classifying an ovarian tumour comprises testing for the presence of chromosomal rearrangement within 2 or more of the RS1 rearrangement hotspots defined in Table 5, e.g. within 3 or more, within 4 or more, within 5 or more, within 6, or within all 7 of the hotspots defined in Table 5.

A set of 6 hotspots may omit any one of the hotspots listed in Table 5, e.g. OV1, OV2, OV3, OV4, OV5, OV6 or OV7.

A set of 5 hotspots may additionally omit any other hotspot listed in Table 5, and so on for smaller sets of hotspots.

For example, a set of 5 hotspots may omit any of the following hotspots:

### OV1 and any one of OV2, OV3, OV4, OV5, OV6 and OV7;

### OV2 and any one of OV1, OV3, OV4, OV5, OV6 and OV7;

### OV3 and any one of OV1, OV2, OV4, OV5, OV6 and OV7;

### OV4 and any one of OV1, OV2, OV3, OV5, OV6 and OV7;

### OV5 and any one of OV1, OV2, OV3, OV4, OV5 and OV7;

### OV6 and any one of OV1, OV2, OV3, OV4, OV5 and OV7;

### OV7 and any one of OV1, OV2, OV3, OV4, OV5 and OV6.

A tumour may be classified as HR-deficient if it has at least one rearrangement within any one of the hotspots tested, e.g. within each of 2 or more, 3 or more, 4 or more, 5 or more, or 6 or more of the hotspots tested.

The term “chromosomal rearrangement” is used to encompass various types of recombination event which may occur within the hotspots defined herein, including tandem duplication, inversion, deletion and translocation.

The presence of any one of these events within a hotspot may constitute a chromosomal rearrangement for the purposes of the invention. The chromosomal rearrangement involved in the “RS1” hotspots identified herein is typically a tandem duplication.

A rearrangement for the purposes of the invention results in the presence of at least one recombination breakpoint within the hotspot, i.e. between the coordinates which define the start and end of the hotspot in Table 1 or 5. A breakpoint is a junction between adjacent sequences which were not adjacent before the recombination event occurred. Thus the methods of the invention may involve determining the presence of one or more breakpoints within the hotspot.

A tandem duplication is a duplication of a particular portion of chromosome, wherein the duplicated portion occurs adjacent to and in the same orientation as the original. Thus, in a chromosomal sequence A-B-C-D-E (shown in an upstream-downstream orientation from left to right), where A, B, C, D and E each represent a block of sequence of (for example) 5 kb, a 10 kb tandem duplication of blocks B and C would result in the chromosomal sequence A-B-C-B-C-D-E. A detectable breakpoint occurs between the upstream copy of block C and the downstream copy of block B.

A deletion results in loss of a particular portion of chromosomal sequence. Thus in the chromosomal sequence A-B-C-D-E, a 5 kb deletion of block C would result in the sequence A-B-D-E, with a single detectable breakpoint between blocks B and D.

An inversion results in a portion of sequence being reversed in orientation. Thus, in the chromosomal sequence A-B-C-D-E, a 10 kb inversion of blocks B and C would result in the sequence A-C′-B′-D-E, where B′ and C′ are in the opposite orientation to the original sequence B-C. Two detectable breakpoints are present, between blocks A and C′, and between blocks B′ and D.

A translocation occurs by exchange of portions of non-homologous chromosomes, and is characterised by one breakpoint on each derivative chromosome.

Tandem duplications, deletions and inversions can be categorised into size groups where the size of a rearrangement is obtained through subtracting the lower breakpoint coordinate from the higher one. Convenient groupings are 1 kb-10 kb, 10 kb-100 kb, 100 kb-1 Mb, 1 Mb-10 Mb, and >10 Mb.

Translocations are the exception and cannot be classified by size.

RS1 hotspots are particularly characterised by tandem duplications, especially of chromosomal fragments of about 1 kb and above, e.g. of about 10 kb and above, often referred to as long tandem repeats. Typically such tandem repeats are from about 1 kb to about 10 Mb in length. (As described above, these may be sub-divided into tandem duplications of 1-10 kb, 10 kb-100 kb; 100 kb-1 Mb, and 1 Mb-10 Mb.)

Thus, tandem duplications of 1 kb and above may be particularly common within the hotspots defined in Tables 1 and 5.

Depending on type, a breakpoint or rearrangement may be identified using some or all of the following parameters:

genome assembly version, lower breakpoint chromosome, lower breakpoint coordinate, higher breakpoint chromosome, higher breakpoint coordinate and either rearrangement class (inversion, tandem duplication deletion, translocation) or strand information of lower and higher breakpoints to enable orientation of rearrangement breakpoints in order to correctly classify them.

The breakpoints may be sorted according to reference genomic coordinate in each sample. The intermutation distance (IMD), defined as the number of base pairs from one rearrangement breakpoint to the one immediately preceding it in the reference genome, may be calculated for each breakpoint.

The presence or absence of chromosomal rearrangement in each tested hotspot is typically determined by comparison with one or more reference sequence(s) for the same hotspot.

Thus the method may comprise determining a data set for each of the tested hotspots from the cancer DNA and comparing each data set from the cancer DNA with a corresponding reference data set to identify any chromosomal rearrangements within each tested hotspot in the cancer DNA (e.g. by identifying a breakpoint within the hotspot).

Thus each data set from the cancer DNA is compared with a corresponding data set derived from a corresponding reference sequence (derived from a reference genome) in order to detect the presence (and optionally type and/or frequency) of rearrangement in the cancer DNA. The content of each data set will depend on the precise format of the particular experiment and the methodology used, but may include full sequence data, copy number of a particular locus or loci (e.g. one or more genes) within the hotspot, absolute or relative positions of particular loci (or pairs of loci). etc.

The reference genome, reference sequence(s) and the reference data set(s) derived therefrom are typically representative of normal (i.e. healthy, non-neoplastic) tissue and may be obtained from any suitable source, including publicly-available or proprietary databases of representative genomic DNA sequences. The reference genome and reference sequence(s) may each be derived from an individual, or may be a compilation or consensus representative of a particular population. The reference genome and reference sequence(s) may be pre-determined, or may be determined as part of the method of the invention, alongside the cancer sample. However, it is generally preferred that the reference genome and reference sequence(s) are derived using DNA (“reference DNA”) from healthy tissue (“reference tissue”) from the same subject, to ensure that any chromosomal rearrangement(s) identified in the cancer is specifically associated with the process of neoplasia and is not a feature of the subject's “normal” genome.

The methods are typically performed on genomic DNA. Genomic DNA from the cancer may be obtained from one or more cells from the cancer (either from peripheral blood or from a biopsy of the cancer) or may be obtained from peripheral blood as free circulating tumour DNA. Reference genomic DNA may be obtained from normal reference tissue, e.g. from the same individual.

In either case, the method may comprise isolating genomic DNA from any samples provided, whether from the cancer or the reference tissue. Whether or not any isolation takes place, the method may comprise further steps of preparing the genomic DNA for analysis. Such preparation steps will depend on the chosen method of analysis and may include fragmentation (by physical or enzymatic means), fractionation, amplification (typically by enzymatic means), enrichment for specific sequences or regions (e.g. hotspots), ligation to adaptors, etc.

Enrichment for hotspot sequences may be carried out by hybridising a sample of fragmented genomic DNA with one or more hybridisation probes each capable of hybridising specifically with a sequence from one of the hotspots to be tested. The DNA which hybridises to the probe or probes is typically isolated from the un-hybridised genomic DNA. Such methods may facilitate the downstream analysis by substantially eliminating sequences from other parts of the genome, leaving only sequences from the hotspots to be tested.

Typically, at least one probe is provided with specificity for each hotspot to be tested. Multiple probes may be provided for each hotspot to be tested. The probes specific for a given hotspot may all have the same sequence or a plurality of different sequences may be provided each capable of hybridising specifically to a different target sequence within the relevant hotspot.

Probes may be provided on solid supports, such as micro-arrays or beads. Any given support may carry a single probe or may carry a plurality of probes. For example, a micro-array may carry a plurality of different probes, each having a defined spatial location on the array. A bead may carry multiple copies of the same probe or a plurality of probes of different sequence.

It may not be necessary in all cases to determine a full sequence of a hotspot in order to identify the presence (or absence) of chromosomal rearrangement, although this may provide the most reliable results, maximising the chance of identifying all informative rearrangements while minimising false positive results. It may be sufficient to determine a sequence (full or partial) of a portion of a hotspot, determine a change in copy number of a particular sequence within a hotspot, or to determine whether a change in distance (chromosomal length) has taken place between selected loci within the hotspot in the cancer DNA as compared to the reference.

Analysis of the DNA from the cancer and, where appropriate, the reference DNA, may be carried out by any suitable method capable of detecting chromosomal rearrangement events, including sequencing and hybridisation methodologies.

Hybridisation-based techniques typically employ microarrays and may involve comparative hybridisation to compare reference and cancer sequences. Suitable techniques include array comparative genomic hybridisation (array CGH).

Suitable sequencing techniques include paired end sequencing (or mate pair sequencing), targeted sequencing, single molecule real-time sequencing, ion semiconductor (Ion Torrent) sequencing, sequencing by synthesis, sequencing by ligation (SOLiD), nano-pore sequencing and pyrosequencing, as well as more traditional techniques of cloning followed by chain termination (Sanger) sequencing.

A number of techniques share a similar approach of sequencing the ends of genomic DNA fragments and comparing the sequences obtained with the corresponding sequences in the reference genome. Thus it is possible to determine whether two particular sequenced portions of genomic DNA are the same distance apart and in the same orientation in the cancer genome and reference genome. Any differences may indicate the presence of chromosomal rearrangement between the two sequenced fragments in the cancer genome.

Such methods typically involve fragmenting genomic DNA and isolating fragments of a selected size. Subsequently, the ends of the selected fragments are linked to adapters containing primer-binding sequences to enable sequencing of the fragment ends. Because the original genomic fragments were selected by size, and the sequenced portions are derived from the ends of those fragments, the separation and orientation of the sequenced portions in the cancer genome is known and can be compared with the corresponding loci in the reference genome.

Various methods are known for linking the ends of the genomic fragments to the adapters. Adapters may be ligated directly to the ends of the genomic fragments. Alternatively, the genomic fragments may be cloned into a vector which comprises suitable adapter sequences flanking the cloning site.

In some methodologies, the end portions of the genomic fragments are themselves isolated from the rest of the genomic fragment and combined into a smaller construct before sequencing. Such constructs may be referred to as “paired end tags” or “di-tags”. The paired end tag typically contains at least 20 nucleotides from each end of the fragment, e.g. at least 21, 22, 23, 24, 25, 26, 27, 28, 29 or at least 30 nucleotides, to provide adequate probability that the sequence is unique in the genome.

Such techniques may employ endonucleases which cut downstream of their recognition sites. Examples include MmeI (which makes a staggered cut 18/20 bases downstream of its recognition site) and EcoP151 and (which makes a staggered cut 25/27 bases downstream of its recognition site). If the adapters used (whether ligated directly to the genomic fragments or flanking a cloning site in a vector) contain recognition sites, the relevant enzyme can be used to create suitable tag sequences which can then be re-ligated into a single paired end tag molecule. If the adapters have been ligated directly to the genomic fragment, the resulting construct will typically be circularised before endonuclease cleavage.

Other methodologies are also available. For example, labelled (e.g. biotinylated) nucleotides may be added to one or both ends of the genomic fragment, followed by circularisation of the labelled genomic fragment, fragmentation of the circularised fragment, and isolation of the labelled fragments (which now contain the ends of the original genomic fragment).

When the ends of the genomic fragments are sequenced directly, without preparation of paired-end tags, the sequencing read length is typically at least 20 nucleotides, at least 50 nucleotides, or at least 100 nucleotides, to increase the chance of the sequence obtained being unique in the genome.

Because of the small amounts of target DNA used, such assays can often be quantitative or semi-quantitative, providing information about copy numbers of particular sequences, as well as simply raw sequence data.

Different types of rearrangement event provide different signatures in such assays. For example, consider a chromosomal sequence A-B-C-D-E, where A, B, C, D and E represent blocks of sequence of (for example) 5 kb, and an assay which employs genomic fragments of 1 kb. Any given fragment could lie wholly within one of A, B, C, D or E, or could span the boundary between two such blocks.

A deletion of block C (yielding the chromosomal sequence A-B-D-E) would result in a loss of sequence signal corresponding to block C from one chromosome, and generation of a novel signal extending from blocks B-D (across the breakpoint) which would previously have been impossible.

By contrast, a tandem duplication of blocks B and C (yielding chromosomal sequence A-B-C-B-C-D-E) would result in an increase in copy number corresponding to blocks C and D from one chromosome, and creation of a novel signal extending from blocks C-B, i.e. across the breakpoint between the upstream and downstream copies of the B-C sequence blocks. There will be no C-B sequence in the reference genome.

Cancers may show multiple chromosomal rearrangements within a given hotspot. Where a hotspot (or portion thereof) exhibits a frequency of rearrangement breakpoints that is at least 10 times greater than the whole genome average density of rearrangements for an individual patient's sample, these rearrangements may be regarded as being “clustered”.

It may be stipulated that a minimum of 10 breakpoints are present in a given region before it can be classified as a cluster of rearrangements. Biologically, the respective partner breakpoint of any rearrangement involved in a clustered region is likely to have arisen at the same mechanistic instant and so can be considered as being involved in the cluster even if located at a distant genomic site according to the reference genome.

Analysis of any given hotspot may involve testing of the entire hotspot, or of a portion thereof. For example, a method may involve analysis of at least 25%, 30%, 35%, 40%, 45%, 50%, 55%, 60%, 65%, 70%, 75%, 80%, 85%, 90% or 95% of any given hotspot.

### Therapeutic Agents

Neoplastic cells (whether breast or ovarian cancers) exhibiting genomic rearrangement events in the identified hotspots are likely to exhibit failure of DNA double strand repair by homologous recombination and may thus be susceptible to therapeutic agents which are more effective against HR-deficient cancers than against HR-proficient cancers. Such agents are referred to in this specification as “agents for treatment of HR-deficient cancers”. This should not be taken to suggest that these agents are only effective against HR-deficient cancers, but simply their efficacy against HR-deficient cancers is greater than against HR-proficient cancers.

Some such agents generate double strand breaks in genomic DNA.

Suitable agents include PARP inhibitors, platinum-based anti-neoplastic agents, anthracyclines, topoisomerase I inhibitors and Wee1 inhibitors.

The enzyme poly-ADP ribose polymerase (PARP) has a key role in DNA repair. Inhibitors of PARP may cause cell death by a variety of mechanisms in HR-deficient cancers. PARP1 inhibitors may be particularly effective. Examples of PARP inhibitors include olaparib (AZD2281), rucaparib (C00338; AG014699; PF01367338), veliparib (ABT888), niraparib (MK4827) and talazoparib (BMN-673). Olaparib, rucaparib and talazoparib may be particularly suitable for the treatment of breast and ovarian cancers.

Platinum-based anti-neoplastic agents (sometimes referred to as “platins”) are coordination complexes of platinum that cause crosslinking of DNA via monoadduct, inter-strand crosslinks, intra-strand crosslinks or DNA protein crosslinks. They may act on the adjacent N-7 position of guanine, forming 1, 2 intra-strand crosslinks. The resultant crosslinking inhibits DNA repair and/or DNA synthesis in cancer cells. Examples include cisplatin, carboplatin, oxaliplatin, nedaplatin, triplatin (BBR3464), phenanthriplatin, picoplatin, lipoplatin and satraplatin (JM216). Carboplatin may be particularly suitable for treatment of breast and ovarian cancers.

Anthracyclines and their derivatives include daunorubicin, doxorubicin, epirubicin, idarubicin, nemorubicin, pixantrone, sabarubicin and valrubicin. Doxorubicin and epirubicin may be particularly useful in the treatment of breast cancer.

Topoisomerase I inhibitors include topotecan, which may be particularly useful for treatment of ovarian cancer.

Wee1 kinase regulates the G2/M checkpoint of mitosis in response to DNA damage. Wee1 inhibitors include AZD1775 (also referred to as MK-1775), PD0166285 (6-(2,6-Dichlorophenyl)-2-[[4-[2-(diethylamino)ethoxy]phenyl]amino]-8-methylpyrido[2,3-d]pyrimidin-7(8H)-one dihydrochloride) and antagonists of Wee1 expression including RNAi, siRNA, antisense RNA and ribozymes specifically directed to Wee1. A Wee1 inhibitor may be used alone or in combination with a further chemotherapeutic agent such as a platin, an anthracycline or a topoisomerase I inhibitor as described above, especially where the further chemotherapeutic agent causes damage to DNA.

Additionally or alternatively, breast cancers which display rearrangement in the hotspot containing ESR1 (designated B23) often have elevated levels of estrogen receptor expression and may be suitable for therapy with agents for treatment of ER-positive cancers. The term “agent for treatment of ER-positive cancers” is used to indicate any agent which has greater efficacy against ER-positive cancers than against ER-negative cancers, and does not necessarily indicate that the agent is only active against ER-positive cancers. Such agents include:
selective estrogen-receptor response modulators (SERMs), such as tamoxifen and toremifene;
aromatase inhibitors, such as anastrozole, exemestane and letrozole;
estrogen-receptor downregulators (ERDs), such as fulvestrant;
luteinizing hormone-releasing hormone agents (LHRHs), such as goserelin, leuprolide and triptorelin.

### EXPERIMENTAL

### Identification of Rearrangement Hotspots

In order to systematically identify hotspots of tandem duplications through the genome, we first considered the background distribution of rearrangements that is known to be non-uniform. A regression analysis was performed to detect and quantify the associations between the distribution of rearrangements and a variety of genomic landmarks including replication time domains, gene-rich regions, background copy number, chromatin state and repetitive sequences (Supplementary materials). The associations learned were taken into consideration creating an adjusted background model and were also applied during simulations, these steps being critical to the following phase of hotspot detection. Adjusted background models and simulated distributions were calculated for RS1 and RS3 tandem duplication signatures separately because of vastly differing numbers of rearrangements in each signature of 5,944 and 13,498 respectively, which could bias the detection of hotspots for the different signatures.

We next employed the principle of intermutation distance¹⁵ (IMD)—the distance from one breakpoint to the one immediately preceding it in the reference genome and used a piecewise constant fitting (PCF) approach^{16,17}, a method of segmentation of sequential data that is frequently utilized in analyses of copy number data. PCF was applied to the IMD of RS1 and RS3 separately, seeking segments of the breast cancer genomes where groups of rearrangements exhibited short IMD, indicative of “hotspots” that are more frequently rearranged than the adjusted background model (Supplementary Materials). The parameters used for the PCF algorithm were optimized against simulated data (Supplementary Materials). We aimed to detect a conservative number of hotspots while minimising the number of false positive hotspots. Note that all highly clustered rearrangements such as those causing driver amplicons had been previously identified in each sample and removed, and thus do not contribute to these hotspots. However, to ensure that a hotspot did not comprise only a few samples with multiple breakpoints each, a minimum of eight samples was required to contribute to each hotspot. Of note, this method negates the use of genomic bins and permits detection of hotspots of varying genomic size.

Thus, the PCF method was applied to RS1 and RS3 rearrangements separately, seeking loci that have a rearrangement density exceeding twice the local adjusted background density for each signature and involving a minimum of eight samples. Interestingly, 0.5% of 13,498 short RS3 tandem duplications contributed towards four RS3 hotspots. By contrast, 10% of 5,944 long RS1 tandem duplications formed 33 hotspots demonstrating that long RS1 tandem duplications are 20 times more likely to form a rearrangement hotspot than short RS3 tandem duplications. Indeed, these were visible as punctuated collections of rearrangements in genome-wide plots of rearrangement breakpoints. RS1 hotspots are shown in Table 1. RS3 hotspots are shown in Table 2.

### Contrasting RS3 Hotspots to RS1 Hotspots

RS3 hotspots had different characteristics to that of RS1 hotspots. The four RS3 hotspots were highly focused, occurred in small genomic windows and exhibited very high rearrangement densities (range 61.8 to 658.3 breakpoints per Mb (FIG. 3B). In contrast, the 33 RS1 hotspots had densities between 7.6 and 83.2 breakpoints per Mb and demonstrated other striking characteristics. In several RS1 hotspots, duplicated segments showed genomic overlap between patients, even when most patients had only one tandem duplication, as depicted in a cumulative plot of duplicated segments for samples contributing rearrangements to a hotspot. Interestingly, the nested tandem duplications that were observed incidentally in the past¹, were a particular characteristic of RS1 hotspots. The hotspots of RS1 and RS3 were distinct from one another apart from one locus where two IncRNAs NEAT1 and MALAT1 reside (discussed in Section 7 of Supplementary Materials).

Assessing the potential genomic consequences of RS1 and RS3 tandem duplications on functional components of the genome¹², RS1 rearrangements were observed to duplicate important driver genes and regulatory elements while RS3 rearrangements were found to mainly transect them (Supplementary materials section 8). This is likely to be related to the size of tandem duplications in these signatures. Short (<10 kb) RS3 tandem duplications are more likely to duplicate very small regions, with the effect equivalent of disrupting genes or regulatory elements. In contrast, RS1 tandem duplications are long (>100 kb), and would be more likely to duplicate whole genes or regulatory elements.

Strikingly, the effects were strongest for tandem duplications that contributed to hotspots of RS1 and RS3 than they were for tandem duplications that were not in hotspots or that were simulated. Thus, although the likelihood of transection/duplication may be governed by the size of tandem duplications, the particular enrichment for hotspots must carry important biological implications.

The enrichment of disruption of tumor suppressor genes by RS3 hotspots (OR 167, P=9.4×10⁻⁴¹ by Fisher's exact test) and is relatively simple to understand—these are likely to be under selective pressure. Accordingly, two of the four RS3 hotspots occurred within well-known tumor suppressors, PTEN and RBI. Other rearrangement classes are also enriched in these genes in-keeping with being driver events (Section 7 of Supplementary Materials).

Furthermore, these sites were identified as putative driver loci in an independent analysis seeking driver rearrangements through gene-based methods¹.

By contrast, the enrichment of oncogene duplication by RS1 hotspots (OR 1.49, P=4.1×10⁻³ by Fisher's exact test) was apparent¹², although not as strong as the enrichment of transections of cancer genes by RS3 hotspots. More notably, the enrichment of other putative regulatory features was also observed. Indeed, we observed that susceptibility loci associated with breast cancer^{8,19} were 4.28 times more frequent in an RS1 hotspot than in the rest of the tandem duplicated genome (P=3.4×10⁻⁴ in Poisson test). Additionally, 18 of 33 (54.5%) RS1 tandem duplication hotspots contained at least one breast super-enhancer.

The density of breast super-enhancers was 3.54 times higher in a hotspot compared to the rest of the tandem duplicated genome (P=7.0×10⁻¹⁶ Poisson test). This effect was much stronger than for non-breast tissue super-enhancers (OR 1.62) or enhancers in general (OR 1.02, Table 3). This gradient reinforces how the relationship between tandem duplication hotspots and regulatory elements deemed as super-enhancer, is tissue-specific.

The reason underlying these observations in RS1 hotspots however is a little less clear. Single or nested tandem duplications in RS1 hotspots effectively increase the number of copies of a genomic region but only incrementally. The enrichment of breast cancer specific susceptibility loci, super-enhancers and oncogenes at hotspots of a very particular mutational signature could reflect an increased likelihood of damage and thus susceptibility to a passenger mutational signature that occurs because of the high transcriptional activity associated with such regions. However, it is also intriguing to consider that the resulting copy number increase could confer some more modest selective advantage and contribute to the driver landscape. To investigate the latter possibility, we explored the impact of RS1 tandem duplications on gene expression.

### Impact of RS1 Hotspots on Expression

Several RS1 hotspots involved validated breast cancer genes¹² (e.g. ESR1, ZNF217) and could conceivably contribute to the driver landscape through increasing the number of copies of a gene—even if by only a single copy.

ESR1 is an example of a breast cancer gene that is a target of an RS1 hotspot. In the vicinity of ESR1 is a breast tissue specific super-enhancer and a breast cancer susceptibility locus. Fourteen samples contribute to this hotspot, of which ten have only a single tandem duplication or simple nested tandem duplications of this site. Six samples had expression data and all showed significantly elevated levels of ESR1 despite modest copy number increase. Four samples have a small number of rearrangements (<30) yet have a highly specific tandem duplication of ESR1, suggestive of selection. Most other samples with rearrangements in the other 32 hotspots were triple negative tumors. By contrast, samples with rearrangements in the ESR1 hotspot showed a different preponderance—eleven of fourteen were estrogen receptor positive tumors. Samples that have tandem duplicated ESR1 even by just a single tandem duplication, have ESR1 expression levels that are in a similar high range as ER positive tumours and are distinctly elevated when compared to the triple negative tumours. Thus we propose that the duplications in the ESR1 hotspot are putative drivers that would not have been detected using customary copy number approaches previously, but are likely to be important to identify because of the associated risk of developing resistance to anti-estrogen chemotherapeutics^{20,21}.

c-MYC encodes a transcription factor that coordinates a diverse set of cellular programs and is deregulated in many different cancer types^{22,23}. 30 patients contributed to the RS1 hotspot at the c-MYC locus with modest copy number gains. A spectrum of genomic outcomes was observed including single or nested tandem duplications, flanking (16 samples) or wholly duplicating the gene body of c-MYC (14 samples). Notably, a breast tissue super-enhancer and two germline susceptibility loci lie in the vicinity of c-MYC^{24 19}. We had a larger number of samples with corresponding RNA-seq data and thus modeled the expression levels of c-MYC taking breast cancer subtype, background copy number (whole chromosome arm gain is common for chr 8) and sought whether tandem duplicating a gene was associated with increased transcription. We find that tandem duplications in the RS1 hotspot were associated with a doubling of the expression level of c-MYC (0.99 s.e. 0.28 log 2 FPKM, P=4.4×10⁻⁴ in t-test) (Table 4).

The expression-related consequences of tandem duplications of putative regulatory elements however, is more difficult to assess because of the uncertainty of the downstream targets of these regulatory elements. Sites enriched for super-enhancers (SENH) may be more highly transcribed and thus exposed to damage including DSB damage. Long tandem duplications are particularly at risk of copying whole genes in contrast to other rearrangement classes. We have thus taken a global gene expression approach and applied a mixed effects model to understand the contribution of tandem duplications of these elements, controlling for breast cancer subtype and background copy number. We find that tandem duplications involving a super-enhancer or breast cancer susceptibility locus are associated with an increase in levels of global gene expression even when the gene itself is not duplicated. The effect is stronger on oncogenes (0.30+−0.20 log 2 FPKM, P=0.12 in likelihood ratio test) than for other genes (0.16 s.e. 0.04 log 2 FPKM, P=1.8×10⁻⁴) within RS1 hotspots or for genes in the rest of the genome (Table 4).

Thus, tandem duplications of cancer genes demonstrate strong expression effects in individual genes (e.g. ESR1 and c-MYC) while tandem duplications of putative regulatory elements demonstrate modest but quantifiable global gene expression effects. The spectrum of functional consequences at these loci could thus range from insignificance, through mild enhancement, to strong selective advantage—consequences of the same somatic rearrangement mutational process.

### Long Tandem Duplication Hotspots are Present and Distinct in Other Cancers

We additionally explored other cancer cohorts where sequence files were available. Two cancer types are known to exhibit tandem duplications, particularly pancreatic and ovarian cancers. Raw sequence files were parsed through our mutation-calling algorithms and rearrangement signatures extracted as for breast cancers. Adjusted background models and simulations were performed on these new datasets separately. The total numbers of available samples (73 ovarian and 96 pancreatic)^{10,11} were much smaller than the breast cancer cohort, which is currently the largest cohort of WGS cancers of a single cancer type in the world. Thus power for detecting hotspots was substantially reduced particularly for pancreatic cancer. Nevertheless, in ovarian tumors 2,923 RS1 rearrangements were found and seven RS1 hotspots identified, of which six were distinct from breast cancer RS1 hotspots. A marked enrichment for ovarian cancer specific super-enhancers (11 super-enhancers over 20.2 Mb, OR 2.9, P=1.9×10⁻³ in Poisson test) was also noted for these hotspots. MUC1, a validated oncogene in ovarian cancer was the focus at one of the hotspots. Thus, although we require larger cohorts of WGS cancers in the future to be definitive, the presentiment is that different cancer-types could have different RS1 hotspots that are focused at highly transcribed sites specific to different tissues.

### Discussion: Selective Susceptibility or Selective Pressure?

Rearrangement signatures may, in principle, be mere passenger read-outs of the stochastic mayhem in cancer cells. However, mutational signatures recurring at specific genomic sites, which also coincide with distinct genomic features, suggest a more directed nature—a sign of either selective susceptibility or selective pressure.

Perhaps it is an attribute of being more highly active or transcribed (e.g. super-enhancers) or some other as yet unknown quality (e.g. germline SNP sites and other hotspots with no discerning features), these hotspots exemplify loci that are rendered more available for DSB damage and more dependent on repair that generates large tandem duplications^{6,25-27}. They signify genomic sites that are innately more susceptible to the HR-deficient tandem duplication mutational process—sites of selective susceptibility.

An alternative argument could also hold true: It could be that the likelihood of damage/repair relating to this mutational process is similar throughout the genome. However, through incrementally increasing the number of copies of coding genes that drive tissue proliferation, survival and invasion (ESR1, ZNF217) or non-coding regions that have minor or intermediate modifying effects in cancer such as germline susceptibility loci or super-enhancer elements, long tandem duplications (unlike other classes of rearrangements) could specifically enhance the overall likelihood of carcinogenesis. The profound implication is that these loci do come under a degree of selective pressure, and that this HR-deficient tandem duplication mutational process is in fact a novel mechanism of generating secondary somatic drivers.

Functional activity related to being a super-enhancer or SNP site could underlie primary susceptibility to mutagenesis of a given locus, but it requires a repair process that generates large tandem duplications to confer selective advantage. Tandem duplication mutagenesis is associated with DSB repair in the context of HR deficiency and is a potentially important mutagenic mechanism driving genetic diversity in evolving cancers by increasing copy number of portions of coding and non-coding genome. It could directly increase the number of copies of an oncogene or alter non-coding sites where super-enhancers/risk loci²⁸ are situated. It could therefore produce a spectrum of driver consequences^{29,30}, ranging from strong effects in coding sequences to weaker effects in the coding and non-coding genome, profoundly, supporting a polygenic model of cancer development.

### Conclusions

Structural mutability in the genome is not uniform. It is influenced by forces of selection and by mutational mechanisms, with recombination-based repair playing a critical role in specific genomic regions. Mutational processes may however not simply be passive contrivances. Some are possibly more harmful than others. We suggest that mutation signatures that confer a high degree of genome-wide variability are potentially more deleterious for somatic cells and thus more clinically relevant. Translational efforts should be focused on identifying and managing these adverse mutational processes in human cancer.

### Supplementary Materials

### Materials and Methods

### 1. Dataset

The primary dataset was obtained from another publication (Nik-Zainal, 2016a). Briefly, 560 matched tumor and normal DNAs were sequenced using Illumina sequencing technology, aligned to the reference genome and mutations called using a suite of somatic mutation calling algorithms as defined previously. In particular, somatic rearrangements were called via BRASS (Breakpoint AnalySiS) (https://github.com/cancerit/BRASS) using discordantly mapping paired-end reads for the discovery phase. Clipped reads were not used to inform discovery. Primary discovery somatic rearrangements were filtered against the germline copy number variants (CNV) in the matched normal, as well as a panel of fifty normal samples from unrelated samples to reduce the likelihood of calling germline CNVs and to reduce the likelihood of calling false positives.

In silico and/or PCR-based validation were performed in a subset of samples (Nik-Zainal, 2016a). Primers were custom-designed and potential rearrangements were PCR-amplified and identified as putatively somatic if a band observed on gel electrophoresis was seen in the tumour and not in the normal, in duplicate. Putative somatic rearrangements were then verified through capillary-sequencing. Amplicons that were successfully sequenced were aligned back to the reference genome using Blat, in order to identify breakpoints to basepair resolution. Alternatively, an in silico analysis was performed using local reassembly. Discordantly mapping read pairs that were likely to span breakpoints as well as a selection of nearby properly paired reads, were grouped for each region of interest. Using the Velvet de novo assembler (Zerbino and Birney, 2008), reads were locally assembled within each of these regions to produce a contiguous consensus sequence of each region. Rearrangements, represented by reads from the rearranged derivative as well as the corresponding non-rearranged allele were instantly recognisable from a particular pattern of five vertices in the de Bruijn graph (a mathematical method used in de novo assembly of (short) read sequences) of component of Velvet. Exact coordinates and features of junction sequence (e.g. microhomology or non-templated sequence) were derived from this, following aligning to the reference genome, as though they were split reads.

Only rearrangements that passed the validation stage were used in these analyses. Furthermore, additional post-hoc filters were included to remove library-related artefacts (creating an excess of inversions in affected samples).

### 2. Rearrangement Signatures

Previously, we had classified rearrangements as mutational signatures as extracted using the Non-Negative Marrix Factorization framework.

Briefly, we first separated rearrangements that were focally clustered from widely dispersed rearrangements because we reasoned that the underlying biological processes that generates these different rearrangement distributions are likely to be distinct. A piecewise constant fitting (PCF) approach was applied in order to distinguish focally clustered rearrangements from dispersed ones. For each sample, both breakpoints of each rearrangement were considered separately from one another and all breakpoints were ordered by chromosomal position. The inter-rearrangement distance, defined as the number of base pairs from one rearrangement breakpoint to the one immediately preceding it in the reference genome, was calculated. Putative regions of clustered rearrangements were identified as having an average inter-rearrangement distance that was at least 10 times greater than the whole genome average for the individual sample. PCF parameters used were γ=25 and kmin=10. The respective partner breakpoint of all breakpoints involved in a clustered region are likely to have arisen at the same mechanistic instant and so were considered as being involved in the cluster even if located at a distant chromosomal site.

In both classes of rearrangements, clustered and non-clustered, rearrangements were subclassified into deletions, inversions and tandem duplications, and then further subclassified according to size of the rearranged segment (1-10 kb, 10 kb-100 kb, 100 kb-1 Mb, 1 Mb-10 Mb, more than 10 Mb). The final category in both groups was interchromosomal translocations. The classification produces a matrix of 32 distinct categories of structural variants across 544 breast cancer genomes. This matrix was decomposed using the previously developed approach for deciphering mutational signatures by searching for the optimal number of mutational signatures that best explains the data (Alexandrov et al., 2013).

In all, six different rearrangement signatures were identified. Rearrangement Signatures 1 and 3 were two signatures that were particularly characterised by tandem duplications.

Rearrangement signature 1 (RS1) is characterized mainly by large tandem duplications (>100 kb) while rearrangement signature 3 (RS3) is characterised mainly by short tandem duplications. There is good reason to believe that these signatures are biologically distinct entities as RS3 is very strongly associated with BRCA1 abrogation (germline or somatic mutation or promoter hypermethylation with concurrent loss of the wild-type allele) while RS1 has not been associated with a specific genetic abnormality.

In order to perform a systematic survey of tandem duplication hotspots, we focused on these two rearrangements signatures. However, tandem duplications (and other rearrangements) are also not uniformly distributed through the genome. Thus, the following sections describe how we detect hotspots of tandem duplications of RS1 and RS3, after correcting for genomic biases.

### 3. Modelling the Background Distribution of Rearrangements

Rearrangements are known to have an uneven distribution in the genome. There have been numerous descriptions linking genomic features such as replication timing with the non-uniform distribution of rearrangements. Thus, any analysis that seeks to detect regions of higher mutability than expected must take the genomic features that influence this non-uniform distribution into account in its background model. In order to formally detect and quantify associations between genomic features and somatic rearrangements in breast cancer, we conducted a multi-variate genome-wide regression analysis.

The genome was divided into non-overlapping genomic bins of 0.5 Mb, and each bin was characterised for the following genomic features:
replication time domain as determined using Repli-Seq data from the MCF7 breast cancer cell line (ENCODE)
gene expression levels
   highly expressed genes (top 25% of genes when ranked by average expression level in our cohort)
   low-expressed genes (remaining 75% of genes)
copy number: average total copy number across the bin in the cohort
repetitive sequences:
   Segmental duplications
   ALU elements
   Other types of repeats
DNAse hyper-sensitive sites (peaks, MCF7, Encode)
Non-mapping sites: N bases in the reference genome
Known fragile sites (Bignell et al., 2010)
Chromatin staining

All of the above features were normalised to a mean of 0 and standard deviation of 1 across the bins for each feature, in order to permit comparability between features. The total number of RS1 and RS3 rearrangement breakpoints were counted for each bin. A regression model was performed in order to learn associated features, using a negative binomial distribution to account for potential over-dispersion.

The model was trained on a total 4,481 bins, after removing the bins containing validated cancer genes. We found that features such as early replication time, highly expressed genes, elevated (general) copy number, DNAse1 hypersensitivity sites and ALU elements were associated with higher densities of RS1 and RS3 rearrangements. They were similarly associated for both tandem duplication signatures although absolute levels of enrichment were only slightly different between the two. Of note, features such as fragile sites, chromatin staining, many classes of repeat elements were neither significantly enriched nor de-enriched for RS1 or RS3 rearrangements.

The properties learned through this regression analysis were then used to perform simulations of rearrangements as described in the next sections, and to calculate the expected number of breakpoints in regions of the genome depending on their features.

Given genomic features of a bin fᵢ (there are N such features) and weights of the negative binomial regression wᵢ, and intercept m, the expected number of breakpoints in a bin given by: bᵢ=e^{m}Πᵢ₌₁^{N}e^{w}^{ᵢ}^{f}^{ᵢ}

In Supplementary Figure S1 we show the exponentiated parameters e^{m} and e^{w}^{ᵢ} fitted by the model, as in this form they have an intuitive multiplicative interpretation. If e^{w}^{ᵢ}=1, the i^{th} genomic feature does not affect the expected number of breakpoints in bins.

### 4. Simulating the Rearrangements

Simulations consisted of as many rearrangements as was observed for each sample in the dataset, preserving the type of rearrangement (tandem duplication, inversion, deletion or translocation), the length of each rearrangement (distance between partner breakpoints) and ensuring that both breakpoints fell within mappable/callable regions in our pipeline.

Simulations also took into account the genomic bias of rearrangements that were identified in Section 3.

In other words, for each rearrangement that was simulated, we:
Drew a position for the lower breakpoint from a genomic bin. Sampling of the lower bin was weighted (non-uniform), with weights proportional to b, the expected number of breakpoint in each bin according to the background model. Within that bin, we uniformly sampled a random genomic position.
Drew the partner breakpoint at an equivalent length as was observed for that rearrangement

The procedure was repeated 10,000 times to build a null distribution. Genomic biases of simulated rearrangements have been confirmed to behave in a similar way to the observed biases.

This null distribution served as the comparator for the next set of analyses, where we used a segmentation algorithm to detect regions that are more mutable than would be expected from our simulations, which correct for the genomic properties that we know influence the uneven distribution of rearrangements.

### 5. Optimization of the PCF Algorithm

The PCF (Piecewise-Constant-Fitting) algorithm is a method of segmentation of sequential data. We used PCF to find segments of the genome that had a much higher rearrangement density than the neighbouring genomic regions, and higher than expected according the background model. We show the significance of the identified hotspots by applying the same method to simulated data (Section 4) that follows the known genomic biases of rearrangements like replication time domains, transcription and background copy number status.

Each rearrangement has two breakpoints and these breakpoints were treated independently of each other. Breakpoints were sorted according to reference genome coordinates and an intermutation distance (IMD) between two genome-sorted breakpoints was calculated for each breakpoint, then log-transformed to base 10. Log 10 IMD were fed into the PCF algorithm.

In order to call a segment of a genome that has a higher rearrangement density as a “hotspot”, a number of parameters had to be determined. The smoothness of segmentation is determined by the gamma (γ) parameter of the PCF analysis. A segment of genome was only considered a peak if it had a sufficient number of mutations, as specified by kₘᵢₙ. The average inter-mutation distance in the segment had to exceed an inter-mutation distance factor (i), which is the threshold when comparing breakpoint density in a segment to genome-wide density of breakpoints:

$$\frac{{d}_{seg}}{{d}_{bg}} > i$$

where:
d_{seg} is the density of breakpoints in a segment defined as:

d_{seg}=(number of breakpoints in segment)/(length in bp of a segment)

d_{bg} is the expected density of breakpoints in the segment, given the background model from Section 3, which includes the genomic covariates of the segment. More specifically, d_{bg}=(Σᵢ₌₁ⁿbᵢ)/(n*s), where bᵢ is the expected number of breakpoints in the bins overlapping the segment, n is the number of overlapping bins, and s is bin size (0.5 Mb).

The choice of parameters kₘᵢₙ, γ and i for the PCF algorithm was based on training on the observed data and comparing the outcomes with that of the simulated data.

Combinations of γ and i were explored to determine the optimal parameters for detection of hotspots where the sensitivity of detection of every hotspot in observed data was balanced against the detection of false positive hotspots in simulated datasets. This was quantified according to the false discovery rate.

Based on the number of detected hotspots on observed and simulated data, we used the γ=8 and i=2 in the final analyses which results in 33 hotspots of RS1 and 4 of RS3. In further 1000 simulated datasets the same parameters resulted on average in 3.3 (standard deviation 1.9) and 0.1 (standard deviation 0.3) hotspots respectively.

A dataset that is not “clean” and that contains a lot of false positive rearrangements, could result in the identification of hotspots of false positives. Thus, it is imperative to have a set of high quality, highly curated rearrangement data—with a better specificity than sensitivity—in order to avoid calling loci where algorithms have a tendency to miscall rearrangements, as hotspots.

6. Workflow Six rearrangement signatures were extracted from this dataset of 560 breast tumours as previously described (Section 2). Each rearrangement was probabilistically assigned to each rearrangement signature given the six rearrangement signatures and the estimated contribution of each signature to each sample (Nik-Zainal, 2016a).

To define hotspots of rearrangements in RS1 and RS3, the PCF algorithm was applied to the log 10 IMD of RS1 or RS3 breakpoints separately using the following parameters: γ=8, kₘᵢₙ=8 and i=2. Each locus was required to be represented by 8 or more samples. The section below describes the hotspots that were identified by this method.

### 7. Identifying Hotspots for Individual Rearrangement Signatures

To explore hotspots associated with signatures of tandem duplications, we first separated rearrangements associated with the two signatures that are strongly characterised by tandem duplications (RS1 and RS3). PCF was performed on each of these two categories. 33 hotspots of long RS1 tandem duplications were identified and 4 hotspots of short RS3 tandem duplications were seen, and they are listed and annotated in Tables 1 and 2 respectively.

We also explored whether the other rearrangement signatures would produce hotspots. Of the six rearrangement signatures, RS4 and RS6 are characterised by interchromosomal and intrachromosomal clustered rearrangements respectively, and RS2 is defined by dispersed interchromosomal rearrangements. RS5 consists mostly of dispersed deletions, mainly shorter than 10 kb.

We hypothesised that distribution of the other rearrangements signatures, particularly the clustered rearrangements, is strongly affected by selection, and we did not build their background models. For these signatures, their genome-wide rearrangement densities served as expected densities in each segment. As hotspots of these signatures the PCF algorithm identified regions with breakpoint density higher than the neighbouring regions and at least twice the genome-wide density. (Hotspots of signatures RS2, RS4, RS5, and RS6 not shown.)

RS4 and RS6 signatures demonstrated 13 hotspots each, 8 of which were overlapping with each other and coincided with various well-described driver amplicons including ERBB2, IGF1R, CCND1, chr8:ZNF703/FGFR1 and ZNF217. Similarly, RS2 demonstrated 21 loci, many of which fell within driver amplicon loci or coincided with known retrotransposition loci. RS5 is characterised by deletion rearrangements and only 3 hotspots were identified, all of which likely represented putative driver loci (PTEN, QKI and TRPS1). RS3 characterised by short tandem duplications also demonstrated 4 hotspots, two were likely drivers (PTEN, RBI) and the significance of the other two are less clear (CDK6 and NEAT1/MALAT1).

Notably, the RS3 hotspot at NEAT1/MALAT1 is the only hotspot that is also an RS1 hotspot. 17 samples contributed to the RS3 hotspot at the site, yet no pattern of effect was noted. Neither MALAT1 nor NEAT1 were transected by the RS3 rearrangements. On the contrary, a clearer pattern was apparent among the samples with RS1 rearrangements. Out of the eight samples that had RS1 rearrangements in the hotspot, we observed a duplication of either NEAT1 or MALAT1 in seven samples. In all eight samples the RS1 duplication spanned one of the three super-enhancers nearby.

Intriguingly, these IncRNAs were also identified as being hotspots for indel and substitution mutagenesis in an experiment searching for putative non-coding drivers (Nik-Zainal, 2016b). We find that the distribution of indel sizes in this region is out-of-keeping with the general distribution of indels in breast cancers. Most were microhomology-mediated indels, which would have commenced as double-strand breaks (DSB) and been fixed latterly by microhomology-mediated end joining mechanisms. NEAT1 and MALAT1 are two of the most highly expressed IncRNAs in breast tissue. Thus, the observation that this is a hotspot of different rearrangement signatures and an indel signature, all of which would have started as DSBs that were eventually fixed using different compensatory DSB repair pathways, would suggest that this is simply a site that is highly exposed to damage. This is likely to be because it is one of the more highly transcribed sites in breast tissue. This interpretation would suggest that the clustering of mutations observed here is not due to selective pressure and that these mutations are not driver events. However, this does not preclude highly significant physiological roles for NEA T1/MALAT1 in the development of cancer. Indeed, it would appear that it is because of the very important biological roles played by NEAT1/MALAT1 that they could be extremely highly transcribed and thus selectively susceptible to DSB mutagenesis.

### 8. Analysis of Effects of Tandem Duplications

We assessed the potential genomic consequences of the two rearrangement signatures associated with tandem duplications on gene function and on regulatory elements.

Rearrangements associated with the RS1 signature are usually long tandem duplications (>100 kb). These are more likely to duplicate whole genes and whole super-enhancer regulatory elements. In contrast, rearrangements associated with the RS3 signature are usually short tandem duplications (<10 kb), and therefore more likely to duplicate smaller regions which could have an effect equivalent of transecting genes or regulatory elements. To formally assess the potential genomic consequences of RS1 and RS3 tandem duplications on gene function and on regulatory elements, we explored the following regulatory elements:
breast cancer susceptibility SNPs
breast-tissue specific super-enhancer regulatory elements
oncogenes (if a duplications covers both a super-enhancer and an oncogene, it will be counted in both categories)
tumour suppressor genes
all genes

An element was considered as wholly duplicated by a tandem duplication if the element was completely between the two breakpoints. An element was considered as transected by a tandem duplication if one or both breakpoints lay within the element.

We did not consider the events where only one breakpoint of duplication was within an element, as the effect of such events on genes and other elements is unclear.

We counted the number of times each of the five elements noted above was duplicated or transected for RS1 and RS3 respectively for:
RS1 or RS3 tandem duplications in hotspots (counted only once per sample—even if there are multiple tandem duplications affecting the same locus in the same person),
RS1 or RS3 tandem duplications that are not within hotspots,
RS1 and RS3 tandem duplications that have been simulated correcting for all the characteristics described above.

Strikingly, RS1 hotspots are clearly enriched for duplicating whole oncogenes and whole super-enhancers, compared to RS1 rearrangements that are not within hotspots and simulated RS1 rearrangements. This enrichment is not observed for RS3 hotspots. Furthermore, RS1 hotspot tandem duplications hardly ever transect genes or regulatory elements. In contrast, RS3 hotspots are strongly enriched for gene transections in-keeping with being driver loci.

Thus here we provide evidence for different genomic consequences—whole gene/regulatory element duplications versus transections—given hotspots generated through different types of rearrangements, long or short tandem duplications.

### 9. Germline Susceptibility Alleles

The list of breast cancer germline susceptibility alleles was derived from the literature (Ahmed et al., 2009; Cox et al., 2007; Easton et al., 2007; Garcia-Closas et al., 2013; Michailidou et al., 2015; Siddiq et al., 2012; Stacey et al., 2008; Thomas et al., 2009; Turnbull et al., 2010; Wei et al., 2016). This analysis is aimed at trying to determine whether there is an enrichment for breast cancer susceptibility SNP alleles in breast cancer, to quantify this relationship and provide a measure of statistical significance.

We performed an analysis that compares the density of SNPs in the genomic footprint of RS1 hotspots against the genomic footprint of other RS1 rearrangements in general (instead of simply to the rest of genome)—this controls for the unevenness in the distribution of tandem duplications. RS1 hotspots encompass 58 Mb of the genome while other segments of the genome covered by (at least) one tandem duplication encompasses 2,106 Mb.

The density of breast cancer susceptibility SNPs outside of RS1 hotspots was 0.036 per Mb. Within RS1 hotspots, there were 9 breast cancer susceptibility SNPs or 0.22 SNPs per Mb. Thus, the odds ratio (OR) of finding a breast cancer susceptibility SNP in RS1 hotspots compared to tandem duplicated regions outside of RS1 hotspots is 4.28 (P=3.4×10⁻⁴ Poisson one-sided).

The Poisson test was used in order to compare rates of events between genomic regions of different sizes, and to account for uncertainty that comes from low number of events (9 SNPs) falling into the hotspots.

### 10. Enrichment for Regulatory Elements

The super-enhancer dataset was obtained from Super-Enhancer Archive (SEA)(Wei et al., 2016). This archive uses publicly available H3K27ac Chip-seq datasets and published super-enhancers lists to produce a comprehensive list of super-enhancers in multiple cell types/tissues. From this list (containing 2,282 unique super-enhancers for 15 human cell types/tissues), we extracted the super-enhancers active in breast cancer (755 elements) and the super-enhancers active in the other cell types/tissues (1,528 elements). Regulatory elements were mutually exclusive to each list to ensure that each super-enhancer was analyzed only in one category, and a super-enhancer was placed in the breast cancer category where there was experimental evidence for multiple activations.

The list of general enhancers was obtained from Ensembl Regulatory Build (GRCh37)(Zerbino et al., 2015). We used the “Multicell” list containing 139,204 elements active in 17 different cell lines. From this list, we filtered out the enhancers that overlapped with super-enhancers, and we obtained a final list composed of 136,858 regulatory elements.

As described in the previous section, we divided the genome into RS1 hotspots (58 Mb), and other segments of the genome covered by a minimum of a single tandem duplication (2,106 Mb). We compared the density of super-enhancers within RS1 hotspot segments and outside of the hotspots.

### Method 1:

The OR of finding a super-enhancer active in breast tissue in RS1 hotspots, compared to regions of the genome rarely covered by RS1 duplications is 3.54 (Poisson one-sided test P=7.0×10⁻¹⁶). The OR for observing a super-enhancers that is not associated with breast tissue is lower at 1.62, with P=6.4×10⁻⁴. The OR for finding any enhancer in an RS1 hotspots is 1.02, with a p-value of 0.12.

### Method 2:

The assumption made in the above analysis is that super-enhancers follow a Poisson distribution, which could be violated by clusters of super-enhancer elements that exist in the genome. We thus performed a set of simulations that do not depend on these assumptions.

In order to assess the likelihood of observing 59 super-enhancers within the regions of RS1 hotspots, the same number of regions of equivalent sizes was sampled from the genome. Similarly as in the previous analysis, the random segments of the genome were drawn from genomic regions representative of non-hotspot tandem duplications (2,106 Mb). The procedure was repeated 10,000 times and super-enhancers falling into the simulated segments were counted.

The observed overlap with 59 or more super-enhancers occurred zero times in 10,000 simulation rounds, by which we estimate the p-value of the observation to be P<10⁻⁴.

### 11. Analysis of Gene Expression

RNA expression levels of genes in the samples were obtained from RNA-seq data as reported by another publication (Nik-Zainal, 2016a).

We set out to assess whether tandem duplications in the hotspots are associated with increased expression of affected genes. However, in many instances, the number of samples contributing to a specific hotspot that also had transcriptomic data was a limiting factor. For example, only six out of fourteen samples that contributed to the ESR1 hotspot had transcriptomic data available.

c-MYC however was a commonly affected locus that had an adequate number of samples (12 samples in the hotspot of which 4 had tandem duplications of the gene itself) to use a linear model to assess the correlation between presence of RS1 tandem duplications at the loci, and the gene expression level, while accounting for different breast receptor expression subtypes (ER positive, triple negative, HER2 positive) and their baseline copy number (background copy number can be variable from one part of the genome to the next e.g. whole arm gains or losses across the genome, or large amplicons). The model was given by:

e˜r+c+t

where
e: gene expression log 2 FPKM
r: receptor type of a sample: ER positive, triple negative, HER2 positive
c: log 2 of background copy number of the gene in individual samples; if the gene itself was tandem duplicated by a dispersed rearrangement, we count the copy number outside of the duplication
t: whether tandem duplications are present in nearby hotspot: TRUE/FALSE The regression model accounts for the variation in gene expression due to amplifications through the parameter c. To establish the effect of tandem duplications on gene expression, we estimate the value of coefficient t.

We obtained the estimates of coefficients in the regression model. We find that the tandem duplications at the c-MYC hotspot are significantly associated with the expression of MYC.

On average, a tandem duplication within the hotspot corresponds to an increase of the gene by 0.99 log 2 FPKM (P=4.4×10⁻⁴ in t-test). In other words, tandem duplications within a c-MYC hotspot were associated with an increase in c-MYC expression level of 2 FPKM (Table 4).

The ability to explore expression effects of tandem duplications of super-enhancers or breast cancer susceptibility SNP loci was limited by the fact that downstream targets of these putative regulatory elements are frequently unknown, uncertain and/or usually involving multiple genes rather than simply a single downstream effector. We thus took a global gene expression approach, to permit detection of expression effects across many genes. This method has its limitations—true signal in some genes may be diluted by the noise from many other genes that are not contributing any signal. However, it does permit detection of effects from many genes simultaneously.

In order to account for between gene variation and tumour subtypes, we used the following mixed-effects linear model:

e˜(1|gene)+(r|gene)+c+d+ds+do

where:
e: gene expression log 2 FPKM
random components:
(1|gene): intercept which is different for each gene
(r|gene): adjustment for receptor type of a sample (ER+, TN, HER2+) which may be
different between genes
fixed components:
c: copy number of the gene in a sample from ASCAT (log 2)
dg: whether the gene was tandem duplicated
ds: whether a super-enhancer or a breast cancer susceptibility locus within 1 Mb of the gene was tandem duplicated (the categories are mutually exclusive, so if a duplication covers both a gene and the super-enhancer, it will appear in the former category only)
do: whether there is some other tandem duplication within 1 Mb

In order to assess the statistical significance of the associations, we also defined two null models. The first one allows us to see and quantify the effects of the tandem duplications of breast cancer super-enhancer or breast cancer susceptibility SNP loci. The first one allows us to see and quantify the effects of tandem duplications of genes themselves.

e˜(1|gene)+(r|gene)+c+dg+do Null model 1:

e˜(1|gene)+(r|gene)+c+ds+do Null model 2:

P-values were obtained by likelihoods ratio tests, between the full and null models, using ANOVA. For fitting the models, we used R and Ime4.

We were able to assess the association between tandem duplications in the hotspots and expression levels of different groups of genes including:
13 putative oncogenes that are implicated in these hotspots: ETV6, MDM2, SRGAP3, WWTR1, FGFR3, WHSC1, MYC, NOTCH1, ESR1, FOXA1, MAML2, ERBB2, ZNF217.
Remaining 509 genes in the hotspots.
A random selection of 489 genes outside of the hotspots We report all of the coefficients of the regression models in Table 4.

In general, tandem duplications in the hotspots were associated with increases in expression levels of nearby genes.
A tandem duplication of an oncogene would be associated with an average increase of expression levels by 0.58 log 2 FPKM (standard error 0.17) (P=6.3×10⁻⁴, by anova test with null model 2).
A tandem duplication of a super-enhancer or regions containing a breast cancer susceptibility SNP proximal to the gene, but not the gene itself, would be associated with an average increase of expression levels of oncogenes by 0.30 (s.e. 0.20) (P=0.12, by comparison with null model 1)
A tandem duplication of any of the remaining 509 genes in the RS1 hotspots (not the oncogenes listed) would be associated with their average increase of expression levels by 0.45 log 2 FPKM (s.e. 0.03) (P=2.2×10⁻¹⁶, null model 2).
A tandem duplication of a super-enhancer or regions containing a breast cancer susceptibility SNP proximal to the gene, but not the gene itself, would be associated with an average increase of expression levels of the 509 genes by 0.16 (s.e. 0.04) (P=1.8×10⁴ by comparison with null model 1).
   12. Hotpots of RS1 in Other Tumours In addition to breast cancer, tumours of other tissue types sometimes show excess of tandem duplications in their genomes. In order to investigate whether the rearrangements in other tumor types also accumulate in hotspots, we utilized previously published sequences of ovarian and pancreatic cancer genomes. We wondered if the hotspots would also co-localize with tissue specific super-enhancers.

We analyzed data from 73 ovarian and 96 pancreatic cancers. Applying the same algorithms as for the breast cancer, we identified 2,923 RS1 rearrangements in ovarian cohort and 448 in pancreatic (compared to 5,944 in breast cancer cohort). In order to assess how many rearrangements are needed to detect hotspots, we randomly sub-sampled the rearrangement dataset from breast cancer.

The results from the simulation matched the number of hotspots detected in ovarian and pancreatic data. We did not find any hotspots in the pancreatic cancer data, and we would have detected none in the breast cancer dataset either, with the same number of tandem duplications as shown in the simulations. However, we were able to identify 7 hotspots of RS1 rearrangements in the ovarian cancer cohort, also consistent with the simulations.

We fitted a background model to the ovarian rearrangements using the copy number data specific to ovarian samples, and applied the PCF algorithm with identical parameters. We identified 7 hotspots of RS1 signature, only one of which coincided with the hotspots we had identified in the breast tumours (RS1_OV_chr3_48.6 Mb). Please refer to Table 5 for the coordinates of the RS1 hotspots in ovarian cancers.

The enrichment of ovarian super-enhancers in the hotspots compared to rest of tandem-duplicated genome was 2.90 fold. MUC1 was focally tandem duplicated in one of the ovarian hotspots (RS1_OV_chr1_150.3 Mb).

### 13. Data Reporting

No statistical methods were used to predetermine sample size. The experiments were not randomised and the investigators were not blinded to allocation during experiments as this was not relevant to the study.

TABLE 1
Table headers:

| | |
|---|---|
| hotspot no. | number of hotspot |
| hotspot.id | ID of hotspot |
| type | PCF-based analysis |
| chr | chromosome |
| start.bp | start coordinate (GRCh37) |
| end.bp | end coordinate (GRCh37) |
| length.bp | size of hotspot |
| number.bps | number of rearrangement breakpoints within a hotspot |
| number.bps.clustered | number of breakpoints of clustered rearrangements in the hotspot (will be 0 |
| | for dispersed rearrangements and number.bps of clustered rearrangements) |
| avgDist.bp | average distance between breakpoints in the hotspot, log10 bp |
| no.samples | number of samples with rearrangements in the hotspot |
| ER.percent | percentage of ER and/or PR positive samples |
| TN.percent | percentage of triple negative samples |
| HER2.percent | percentage of HER2 positive samples |
| segment.density | number.bps/length.bp |
| factor | segment.density/genome wide density of rearrangements |
| d.bg | expected density of breakpoints according to the background model |
| d.obs.exp | segment.density/d.bg |
| fragileSites | fragile sites |
| transposons | IDs of L1 transposon sites coinciding with the hotspot. |
| genes | list of genes coinciding with the hotspot |
| targ.genes | gene hit most frequently by rearrangements (compared to rest of hotspot, |
| | binomial test, Poisson distribution). |
| targ.genes.2 | gene hit most frequently by rearrangements (compared to flanking |
| | sequence of gene, window size 10 kb, binomial test, Poisson distribution). |
| censusGenes | list of cancer census genes within the hotspot (downloaded from COSMIC |
| | XX) |
| targ.census.genes | intersection of targ.genes and censusGenes |
| breastGenes | list of breast cancer genes |
| figure.label | included in figure as label |
| amplified.dom | list of genes within 5 mb of the hotspot of clustered rearrangements, |
| | classified as dominant in the census, sorted by frequency across the |
| | samples |
| BreastSNPs | breast cancer susceptibility SNPs that overlap with the hotspot |
| superenhancers | super-enhancers that overlap with the hotspot |

| hotspot no. | hotspot.id | type | chr | start.bp | end.bp | length.bp |
|---|---|---|---|---|---|---|
| B1 | peak_RS1_chr1_0.7mb | RS1 | 1 | 735,890 | 1,700,712 | 964,822 |
| B2 | peak_RS1_chr1_66.7mb | RS1 | 1 | 66,699,372 | 67,093,762 | 394,390 |
| B3 | peak_RS1_chr1_234.6mb | RS1 | 1 | 234,643,138 | 235,822,749 | 1,179,611 |
| B4 | peak_RS1_chr12_11.8mb | RS1 | 12 | 11,841,798 | 12,846,639 | 1,004,841 |
| B5 | peak_RS1_chr12_69mb | RS1 | 12 | 69,007,914 | 70,453,514 | 1,445,600 |
| B6 | peak_RS1_chr12_75.9mb | RS1 | 12 | 75,854,477 | 76,521,353 | 666,876 |
| B7 | peak_RS1_chr12_98.9mb | RS1 | 12 | 98,903,996 | 102,195,693 | 3,291,697 |
| B8 | peak_RS1_chr3_3.3mb | RS1 | 3 | 3,328,620 | 11,049,419 | 7,720,799 |
| B9 | peak_RS1_chr3_47.1mb | RS1 | 3 | 47,101,952 | 52,489,018 | 5,387,066 |
| B10 | peak_RS1_chr3_148.2mb | RS1 | 3 | 148,151,259 | 149,404,706 | 1,253,447 |
| B11 | peak_RS1_chr4_0.4mb | RS1 | 4 | 379,209 | 2,799,228 | 2,420,019 |
| B12 | peak_RS1_chr4_91.1mb | RS1 | 4 | 91,127,738 | 92,819,714 | 1,691,976 |
| B13 | peak_RS1_chr5_36.7mb | RS1 | 5 | 36,703,047 | 37,327,012 | 623,965 |
| B14 | peak_RS1_chr5_44.2mb | RS1 | 5 | 44,222,786 | 44,801,720 | 578,934 |
| B15 | peak_RS1_chr8_1.6mb | RS1 | 8 | 1,598,492 | 4,977,627 | 3,379,135 |
| B16 | peak_RS1_chr8_67.2mb | RS1 | 8 | 67,246,539 | 67,673,469 | 426,930 |
| B17 | peak_RS1_chr8_89.6mb | RS1 | 8 | 89,577,063 | 90,909,008 | 1,331,945 |
| B18 | peak_RS1_chr8_116.6mb | RS1 | 8 | 116,610,790 | 117,921,508 | 1,310,718 |
| B19 | peak_RS1_chr8_127.8mb | RS1 | 8 | 127,848,258 | 129,291,461 | 1,443,203 |
| B20 | peak_RS1_chr8_141.3mb | RS1 | 8 | 141,343,280 | 142,586,054 | 1,242,774 |
| B21 | peak_RS1_chr9_139.4mb | RS1 | 9 | 139,425,188 | 140,379,689 | 954,501 |
| B22 | peak_RS1_chr6_107mb | RS1 | 6 | 106,965,583 | 107,313,692 | 348,109 |
| B23 | peak_RS1_chr6_151.8mb | RS1 | 6 | 151,753,959 | 152,601,611 | 847,652 |
| B24 | peak_RS1_chr2_182.8mb | RS1 | 2 | 182,826,713 | 187,430,553 | 4,603,840 |
| B25 | peak_RS1_chr7_69.6mb | RS1 | 7 | 69,612,910 | 70,268,704 | 655,794 |
| B26 | peak_RS1_chr14_37.9mb | RS1 | 14 | 37,932,706 | 39,251,820 | 1,319,114 |
| B27 | peak_RS1_chr14_67.7mb | RS1 | 14 | 67,662,099 | 70,360,290 | 2,698,191 |
| B28 | peak_RS1_chr11_34.5mb | RS1 | 11 | 34,462,630 | 34,978,273 | 515,643 |
| B29 | peak_RS1_chr11_65.2mb | RS1 | 11 | 65,197,499 | 65,341,680 | 144,181 |
| B30 | peak_RS1_chr11_95.6mb | RS1 | 11 | 95,590,911 | 96,020,456 | 429,545 |
| B31 | peak_RS1_chr17_26.8mb | RS1 | 17 | 26,810,266 | 28,071,299 | 1,261,033 |
| B32 | peak_RS1_chr17_37.7mb | RS1 | 17 | 37,678,390 | 37,975,805 | 297,415 |
| B33 | peak_RS1_chr20_47.4mb | RS1 | 20 | 47,380,412 | 53,063,961 | 5,683,549 |

| hotspot no. | number.bps | number.bps.clustered | avgDist.bp | no.samples |
|---|---|---|---|---|
| B1 | 22 | 0 | 4.43817918 | 11 |
| B2 | 26 | 0 | 3.89988317 | 11 |
| B3 | 27 | 0 | 4.4471482 | 15 |
| B4 | 55 | 0 | 3.85940662 | 18 |
| B5 | 24 | 0 | 4.48071031 | 13 |
| B6 | 15 | 0 | 4.43052935 | 8 |
| B7 | 48 | 0 | 4.52303685 | 27 |
| B8 | 71 | 0 | 4.74613691 | 32 |
| B9 | 71 | 0 | 4.5837879 | 28 |
| B10 | 25 | 0 | 4.4094739 | 15 |
| B11 | 33 | 0 | 4.54054565 | 17 |
| B12 | 25 | 0 | 4.49659162 | 15 |
| B13 | 13 | 0 | 4.25610574 | 8 |
| B14 | 24 | 0 | 4.0709807 | 13 |
| B15 | 37 | 0 | 4.69500015 | 14 |
| B16 | 18 | 0 | 4.15677472 | 9 |
| B17 | 24 | 0 | 4.34254169 | 12 |
| B18 | 46 | 0 | 4.14393972 | 20 |
| B19 | 68 | 0 | 3.98405426 | 30 |
| B20 | 34 | 0 | 4.30646886 | 18 |
| B21 | 20 | 0 | 4.4027788 | 9 |
| B22 | 12 | 0 | 4.02369316 | 8 |
| B23 | 28 | 0 | 4.25704596 | 14 |
| B24 | 35 | 0 | 4.63549637 | 18 |
| B25 | 19 | 0 | 4.04470239 | 13 |
| B26 | 17 | 0 | 4.0879059 | 13 |
| B27 | 41 | 0 | 4.58596207 | 21 |
| B28 | 28 | 0 | 4.03041486 | 12 |
| B29 | 12 | 0 | 3.88158014 | 8 |
| B30 | 12 | 0 | 4.13455709 | 9 |
| B31 | 41 | 0 | 4.07229402 | 17 |
| B32 | 17 | 0 | 3.8614682 | 11 |
| B33 | 81 | 0 | 4.49210435 | 31 |

| hotspot no. | ER.percent | TN.percent | HER2.percent | segment.density | factor |
|---|---|---|---|---|---|
| B1 | 45 | 45 | 9 | 2.28E−05 | 5.4 |
| B2 | 9 | 82 | 0 | 6.59E−05 | 15.5 |
| B3 | 27 | 60 | 13 | 2.29E−05 | 5.4 |
| B4 | 0 | 89 | 11 | 5.47E−05 | 12.9 |
| B5 | 31 | 46 | 23 | 1.66E−05 | 3.9 |
| B6 | 25 | 75 | 0 | 2.25E−05 | 5.3 |
| B7 | 11 | 74 | 15 | 1.46E−05 | 3.4 |
| B8 | 25 | 66 | 9 | 9.20E−06 | 2.2 |
| B9 | 21 | 57 | 21 | 1.32E−05 | 3.1 |
| B10 | 7 | 93 | 0 | 1.99E−05 | 4.7 |
| B11 | 24 | 59 | 18 | 1.36E−05 | 3.2 |
| B12 | 13 | 73 | 13 | 1.48E−05 | 3.5 |
| B13 | 25 | 63 | 13 | 2.08E−05 | 4.9 |
| B14 | 85 | 8 | 8 | 4.15E−05 | 9.8 |
| B15 | 14 | 57 | 29 | 1.09E−05 | 2.6 |
| B16 | 0 | 89 | 11 | 4.22E−05 | 9.9 |
| B17 | 17 | 67 | 17 | 1.80E−05 | 4.2 |
| B18 | 20 | 60 | 20 | 3.51E−05 | 8.3 |
| B19 | 20 | 67 | 17 | 4.71E−05 | 11.1 |
| B20 | 17 | 78 | 11 | 2.74E−05 | 6.4 |
| B21 | 11 | 78 | 11 | 2.10E−05 | 4.9 |
| B22 | 13 | 63 | 25 | 3.45E−05 | 8.1 |
| B23 | 79 | 14 | 7 | 3.30E−05 | 7.8 |
| B24 | 17 | 56 | 28 | 7.60E−06 | 1.8 |
| B25 | 31 | 62 | 8 | 2.90E−05 | 6.8 |
| B26 | 54 | 15 | 31 | 1.29E−05 | 3.0 |
| B27 | 14 | 67 | 19 | 1.52E−05 | 3.6 |
| B28 | 8 | 92 | 0 | 5.43E−05 | 12.8 |
| B29 | 0 | 88 | 13 | 8.32E−05 | 19.6 |
| B30 | 11 | 78 | 11 | 2.79E−05 | 6.6 |
| B31 | 12 | 65 | 24 | 3.25E−05 | 7.6 |
| B32 | 18 | 36 | 45 | 5.72E−05 | 13.4 |
| B33 | 29 | 58 | 10 | 1.43E−05 | 3.4 |

| hotspot no. | d.bg | d.obs.exp | fragileSites |
|---|---|---|---|
| B1 | 5.68E−06 | 4.0 | FRA1A |
| B2 | 3.29E−06 | 20.0 | |
| B3 | 8.32E−06 | 2.8 | FRA1H |
| B4 | 5.65E−06 | 9.7 | |
| B5 | 5.45E−06 | 3.0 | |
| B6 | 4.03E−06 | 5.6 | |
| B7 | 4.72E−06 | 3.1 | |
| B8 | 4.05E−06 | 2.3 | |
| B9 | 6.02E−06 | 2.2 | |
| B10 | 4.88E−06 | 4.1 | FRA3D |
| B11 | 5.89E−06 | 2.3 | |
| B12 | 4.62E−06 | 3.2 | FRA4F FRA4F-narrow |
| B13 | 6.37E−06 | 3.3 | FRA5A FRA5E-narrow |
| B14 | 3.20E−06 | 12.9 | |
| B15 | 2.30E−06 | 4.8 | |
| B16 | 7.91E−06 | 5.3 | FRA8F |
| B17 | 4.84E−06 | 3.7 | |
| B18 | 1.11E−05 | 3.2 | FRA8C; FRA8E |
| B19 | 7.37E−06 | 6.4 | FRA8C-narrow |
| B20 | 1.00E−05 | 2.7 | FRA8D |
| B21 | 6.39E−06 | 3.3 | |
| B22 | 5.74E−06 | 6.0 | FRA6F |
| B23 | 6.91E−06 | 4.8 | |
| B24 | 3.29E−06 | 2.3 | FRA2G; FRA2H |
| B25 | 4.66E−06 | 6.2 | FRA7J |
| B26 | 3.53E−06 | 3.7 | |
| B27 | 6.02E−06 | 2.5 | FRA14B FRA14C-narrow; |
| | | | FRA14C FRA14C-narrow |
| B28 | 5.67E−06 | 9.6 | FRA11E |
| B29 | 7.01E−06 | 11.9 | FRA11H |
| B30 | 2.21E−06 | 12.6 | |
| B31 | 7.16E−06 | 4.5 | |
| B32 | 1.65E−05 | 3.5 | |
| B33 | 6.02E−06 | 2.4 | |

| hotspot no. | genes |
|---|---|
| B1 | SAMD11; NOC2L; KLHL17; PLEKHN1; C1orf170; HES4; ISG15; AGRN; RNF223; C1orf159; |
| | TTLL10; TNFRSF18; TNFRSF4; SDF4; B3GALT6; FAM132A; UBE2J2; SCNN1D; ACAP3; |
| | PUSL1; CPSF3L; GLTPD1; TAS1R3; DVL1; MXRA8; AURKAIP1; CCNL2; MRPL20; ANKRD65; |
| | TMEM88B; VWA1; ATAD3C; ATAD3B; ATAD3A; TMEM240; SSU72; AL645728.1; |
| | C1orf233; MIB2; MMP23B; CDK11B; SLC35E2B; CDK11A; SLC35E2; NADK |
| B2 | PDE4B; SGIP1 |
| B3 | IRF2BP2; TOMM20; RBM34; ARID4B; GGPS1; TBCE; B3GALNT2; GNG4 |
| B4 | ETV6; BCL2L14; LRP6; MANSC1; LOH12CR2; LOH12CR1; DUSP16; CREBL2; GPR19 |
| B5 | RAP1B; NUP107; SLC35E3; MDM2; CPM; CPSF6; LYZ; YEATS4; FRS2; CCT2; LRRC10; |
| | BEST3; RAB3IP; MYRFL |
| B6 | GLIPR1; KRR1; PHLDA1; NAP1L1 |
| B7 | TMPO; SLC25A3; IKBIP; APAF1; ANKS1B; FAM71C; UHRF1BP1L; ACTR6; DEPDC4; SCYL2; |
| | SLC17A8; NR1H4; GAS2L3; ANO4; SLC5A8; UTP20; ARL1; SPIC; MYBPC1; CHPT1; |
| | SYCP3; GNPTAB |
| B8 | SUMF1; LRRN1; SETMAR; ITPR1; BHLHE40; ARL8B; AC026202.1; EDEM1; GRM7; LMCD1; |
| | SSUH2; CAV3; OXTR; RAD18; SRGAP3; THUMPD3; SETD5; LHFPL4; MTMR14; CPNE9; |
| | BRPF1; OGG1; CAMK1; TADA3; ARPC4; ARPC4-TTLL3; TTLL3; RPUSD3; CIDEC; JAGN1; |
| | IL17RE; IL17RC; CRELD1; PRRT3; EMC3; FANCD2; FANCD2OS; BRK1; VHL; IRAK2; |
| | TATDN2; GHRL; SEC13; ATP2B2; SLC6A11; SLC6A1 |
| B9 | SETD2; KIF9; KLHL18; PTPN23; SCAP; ELP6; CSPG5; SMARCC1; DHX30; MAP4; CDC25A; |
| | CAMP; ZNF589; NME6; SPINK8; FBXW12; PLXNB1; CCDC51; TMA7; ATRIP; TREX1; |
| | SHISA5; PFKFB4; UCN2; COL7A1; UQCRC1; TMEM89; SLC26A6; CELSR3; NCKIPSD; IP6K2; |
| | PRKAR2A; SLC25A20; ARIH2OS; ARIH2; P4HTM; WDR6; DALRD3; NDUFAF3; IMPDH2; |
| | QRICH1; QARS; USP19; LAMB2; CCDC71; KLHDC8B; C3orf84; CCDC36; C3orf62; |
| | USP4; GPX1; RHOA; TCTA; AMT; NICN1; DAG1; BSN; APEH; MST1; RNF123; AMIGO3; |
| | GMPPB; IP6K1; CDHR4; FAM212A; UBA7; TRAIP; CAMKV; MST1R; MON1A; RBM6; RBM5; |
| | SEMA3F; GNAT1; GNAI2; LSMEM2; IFRD2; HYAL3; NAT6; HYAL1; HYAL2; TUSC2; RASSF1; |
| | ZMYND10; NPRL2; CYB561D2; TMEM115; CACNA2D2; C3orf18; HEMK1; CISH; |
| | MAPKAPK3; DOCK3; MANF; RBM15B; VPRBP; RAD54L2; TEX264; GRM2; IQCF6; IQCF3; |
| | IQCF2; IQCF5; IQCF1; RRP9; PARP3; GPR62; PCBP4; ABHD14B; ABHD14A; ACY1; |
| | ABHD14A-ACY1; RPL29; DUSP7; LINC00696; POC1A; ALAS1; TLR9; TWF2; PPM1M; WDR82; |
| | GLYCTK; DNAH1; BAP1; PHF7; SEMA3G; TNNC1 |
| B10 | AGTR1; CPB1; CPA3; GYG1; HLTF; HPS3; CP; TM4SF18; TM4SF1; TM4SF4; WWTR1 |
| B11 | ZNF721; PIGG; PDE6B; ATP5I; MYL5; MFSD7; PCGF3; CPLX1; GAK; TMEM175; DGKQ; |
| | SLC26A1; IDUA; FGFRL1; RNF212; SPON2; CTBP1; MAEA; UVSSA; CRIPAK; NKX1-1; |
| | FAM53A; SLBP; TMEM129; TACC3; FGFR3; LETM1; WHSC1; NELFA; C4orf48; NAT8L; |
| | POLN; HAUS3; MXD4; ZFYVE28; RNF4; FAM193A; TNIP2; SH3BP2 |
| B12 | CCSER1 |
| B13 | NIPBL; C5orf42; NUP155 |
| B14 | FGF10 |
| B15 | DLGAP2; CLN8; ARHGEF10; KBTBD11; MYOM2; CSMD1 |
| B16 | RRS1; ADHFE1; C8orf46; MYBL1; VCPIP1; C8orf44; SGK3 |
| B17 | RIPK2 |
| B18 | TRPS1; EIF3H; UTP23; RAD21 |
| B19 | POU5F1B; MYC; TMEM75 |
| B20 | TRAPPC9; CHRAC1; AGO2; PTK2; DENND3; SLC45A4; GPR20; PTP4A3 |
| B21 | NOTCH1; EGFL7; AGPAT2; FAM69B; LCN10; LCN6; LCN8; LCN15; TMEM141; CCDC183; |
| | RABL6; C9orf172; PHPT1; MAMDC4; EDF1; TRAF2; FBXW5; C8G; LCN12; C9orf141; PTGDS; |
| | LCNL1; C9orf142; CLIC3; ABCA2; C9orf139; FUT7; NPDC1; ENTPD2; SAPCD2; UAP1L1; |
| | AL807752.1; MAN1B1; DPP7; GRIN1; LRRC26; TMEM210; ANAPC2; SSNA1; TPRN; |
| | TMEM203; NDOR1; RNF208; C9orf169; RNF224; SLC34A3; TUBB4B; FAM166A; C9orf173; |
| | NELFB; TOR4A; NRARP; EXD3; NOXA1; ENTPD8; NSMF; PNPLA7 |
| B22 | AIM1; RTN4IP1; QRSL1 |
| B23 | RMND1; C6orf211; CCDC170; ESR1; SYNE1 |
| B24 | PPP1R1C; PDE1A; DNAJC10; FRZB; NCKAP1; DUSP19; NUP35; ZNF804A; FSIP2; ZC3H15 |
| B25 | AUTS2 |
| B26 | MIPOL1; FOXA1; TTC6; SSTR1; CLEC14A |
| B27 | FAM71D; MPP5; ATP6V1D; EIF2S1; PLEK2; TMEM229B; PLEKHH1; PIGH; ARG2; VTI1B; |
| | RDH11; RDH12; ZFYVE26; RAD51B; ZFP36L1; ACTN1; DCAF5; EXD2; GALNT16; ERH; |
| | SLC39A9; PLEKHD1; CCDC177; CCDC177; KIAA0247; SRSF5; SLC10A1; SMOC1 |
| B28 | CAT; ELF5; EHF; APIP; PDHX |
| B29 | SCYL1; LTBP3; SSSCA1; FAM89B |
| B30 | MTMR2; MAML2 |
| B31 | RP11-192H23.4; SLC13A2; FOXN1; UNC119; PIGS; ALDOC; SPAG5; SGK494; KIAA0100; SDF2; |
| | SUPT6H; PROCA1; RAB34; RPL23A; TLCD1; NEK8; TRAF4; FAM222B; ERAL1; FLOT2; |
| | DHRS13; PHF12; PIPOX; SEZ6; MYO18A; TIAF1; CRYBA1; NUFIP2; TAOK1; ABHD15; TP53I13; |
| | GIT1; ANKRD13B; CORO6; SSH2 |
| B32 | CDK12; NEUROD2; PPP1R1B; STARD3; TCAP; PNMT; PGAP3; ERBB2; MIEN1; GRB7; IKZF3 |
| B33 | PREX1; ARFGEF2; CSE1L; STAU1; DDX27; ZNFX1; KCNB1; PTGIS; B4GALT5; SLC9A8; |
| | SPATA2; RNF114; SNAI1; TMEM189-UBE2V1; UBE2V1; TMEM189; CEBPB; PTPN1; FAM65C; |
| | PARD6B; BCAS4; ADNP; DPM1; MOCS3; KCNG1; NFATC2; ATP9A; SALL4; ZFP64; TSHZ2; |
| | ZNF217; BCAS1; CYP24A1; PFDN4 |

| hotspot no. | targ.genes | censusGenes | targ.census.genes | breast Genes | breastSNPs | superenhancers |
|---|---|---|---|---|---|---|
| B1 | UBE2J2 | | | | | SENH-RNF223- |
| | | | | | | chr1: 1005293 |
| B2 | PDE4B; SGIP1 | | | | | SENH-PDE4B- |
| | | | | | | chr1: 66712370; |
| | | | | | | SENH-PDE4B- |
| | | | | | | chr1: 66778961 |
| B3 | | | | | | SENH-IRF2BP2- |
| | | | | | | chr1: 234709673; |
| | | | | | | SENH-LINC01132- |
| | | | | | | chr1: 234857710; |
| | | | | | | SENH-LINC01132- |
| | | | | | | chr1: 234907393; |
| | | | | | | SENH-LOC101927851- |
| | | | | | | chr1: 235010375; |
| | | | | | | SENH-LOC101927851- |
| | | | | | | chr1: 235068890; |
| | | | | | | SENH-TOMM20- |
| | | | | | | chr1: 235242252 |
| B4 | ETV6; BCL2L14; | ETV6 | ETV6 | | | SENH-ETV6- |
| | LRP6 | | | | | chr12: 11949422; |
| | | | | | | SENH-BCL2L14- |
| | | | | | | chr12: 12161273 |
| B5 | RAP1B; NUP107; | MDM2 | | | | |
| | CPSF6; YEATS4; | | | | | |
| | BEST3 | | | | | |
| B6 | NAP1L1 | | | | | SENH-PHLDA1- |
| | | | | | | chr12: 76405800 |
| B7 | IKBIP | | | | | |
| B8 | ITPR1; SEC13; | SRGAP3; | | VHL | rs6762644 | SENH-EGOT- |
| | SLC6A1 | FANCD2; VHL | | | | chr3: 4780474; |
| | | | | | | SENH-BHLHE40- |
| | | | | | | chr3: 5027453 |
| B9 | PLXNB1 | SETD2; BAP1 | | SETD2; | | SENH-SEMA3F- |
| | | | | BAP1 | | chr3: 50194710; |
| | | | | | | SENH-GNAI2- |
| | | | | | | chr3: 50264852; S |
| | | | | | | ENH-CISH- |
| | | | | | | chr3: 50625949; S |
| | | | | | | ENH-DUSP7- |
| | | | | | | chr3: 52079728 |
| B10 | | WWTR1 | | | | |
| B11 | DGKQ; LETM1; | FGFR3; WHSC1 | | FGFR3 | | SENH-SH3BP2- |
| | SH3BP2 | | | | | chr4: 2792408 |
| B12 | CCSER1 | | | | | |
| B13 | | | | | | |
| B14 | FGF10 | | | | rs10941679 | |
| B15 | | | | | | |
| B16 | | | | | | SENH-C8orf46- |
| | | | | | | chr8: 67433991 |
| B17 | RIPK2 | | | | | |
| B18 | | RAD21 | | RAD21 | rs13267382 | |
| B19 | MYC | MYC | MYC | | rs13281615; | SENH-CCAT1- |
| | | | | | rs11780156 | chr8: 128196669; |
| | | | | | | SENH-CASC21- |
| | | | | | | chr8: 128305149; |
| | | | | | | SENH-CCAT2- |
| | | | | | | chr8:128403573 |
| B20 | | | | | | SENH-DENND3- |
| | | | | | | chr8: 142129714; |
| | | | | | | SENH-SLC45A4- |
| | | | | | | chr8: 142237099 |
| B21 | LRRC26 | NOTCH1 | | NOTCH1 | | SENH-LINC01573- |
| | | | | | | chr9: 139427472 |
| B22 | | | | | | |
| B23 | | | | ESR1 | rs2046210; | SENH-ARMT1- |
| | | | | | rs12662670 | chr6: 151803491 |
| B24 | ZC3H15 | | | | | |
| B25 | AUTS2 | | | | | |
| B26 | MIPOL1; | FOXA1 | | FOXA1 | | SENH-FOXA1- |
| | TTC6 | | | | | chr14: 38052956 |
| B27 | | | | | rs999737; | SENH-RAD51B- |
| | | | | | rs2588809 | chr14: 68604521; |
| | | | | | | SENH-RAD51B- |
| | | | | | | chr14: 68864007; |
| | | | | | | SENH-RAD51B- |
| | | | | | | chr14: 68925660; |
| | | | | | | SENH-ZFP36L1- |
| | | | | | | chr14: 68961774; |
| | | | | | | SENH-ZFP36L1- |
| | | | | | | chr14: 69010932; |
| | | | | | | SENH-ZFP36L1- |
| | | | | | | chr14: 69143232; |
| | | | | | | SENH-ZFP36L1- |
| | | | | | | chr14: 69224405; |
| | | | | | | SENH-ZFP36L1- |
| | | | | | | chr14: 69281323; |
| | | | | | | SENH-ACTN1-AS1- |
| | | | | | | chr14: 69417780; |
| | | | | | | SENH-DCAF5- |
| | | | | | | chr14: 69507227 |
| B28 | CAT; ELF5; | | | | | |
| | EHF; PDHX | | | | | |
| B29 | | | | | | SENH-MALAT1- |
| | | | | | | chr11: 65238917; |
| | | | | | | SENH-SSSCA1-AS1- |
| | | | | | | chr11: 65323331 |
| B30 | MTMR2; | MAML2 | MAML2 | | | SENH-MAML2- |
| | MAML2 | | | | | chr11: 95888692; |
| | | | | | | SENH-MAML2- |
| | | | | | | chr11: 95963875 |
| B31 | | | | | | |
| B32 | CDK12; | CDK12; | CDK12 | CDK12; | | |
| | IKZF3 | ERBB2 | | ERBB2 | | |
| B33 | ZNF217 | | | | | SENH-PREX1- |
| | | | | | | chr20: 47367806; |
| | | | | | | SENH-PREX1- |
| | | | | | | chr20: 47434951; |
| | | | | | | SENH-PREX1- |
| | | | | | | chr20: 47463345; |
| | | | | | | SENH-PTGIS- |
| | | | | | | chr20: 48200728; |
| | | | | | | SENH-B4GALT5- |
| | | | | | | chr20: 48285223; |
| | | | | | | SENH-B4GALT5- |
| | | | | | | chr20: 48315836; |
| | | | | | | SENH-SLC9A8- |
| | | | | | | chr20: 48381625; |
| | | | | | | SENH-CEBPB- |
| | | | | | | chr20: 48804007; |
| | | | | | | SENH-LINC01272- |
| | | | | | | chr20: 48869312; |
| | | | | | | SENH-PTPN1- |
| | | | | | | chr20: 49047228; |
| | | | | | | SENH-NFATC2- |
| | | | | | | chr20: 50097057; |
| | | | | | | SENH-ZNF217- |
| | | | | | | chr20: 52195343; |
| | | | | | | SENH-ZNF217- |
| | | | | | | chr20: 52238527; |
| | | | | | | SENH-SUMO1P1- |
| | | | | | | chr20: 52346173; |
| | | | | | | SENH-SUMO1P1- |
| | | | | | | chr20: 52444580; |
| | | | | | | SENH-SUMO1P1- |
| | | | | | | chr20: 52516053; |
| | | | | | | SENH-CYP24A1- |
| | | | | | | chr20: 52726626 |

| hotspot no. | Samples |
|---|---|
| B1 | PD4315a; PD4953a; PD5956a; PD11368a; PD11379a; PD5935a; PD7066a; PD9604a; |
| | PD18024a; PD4841a; PD22355a |
| B2 | PD11743a; PD4956a; PD5930a; PD5935a; PD7248a; PD7316a; PD7426a; PD9571a; |
| | PD11748a; PD22363a; PD23559a |
| B3 | PD4976a; PD8978a; PD9464a; PD13312a; PD6722a; PD7066a; PD8660a2; PD8964a; |
| | PD13297a; PD13165a; PD7304a; PD3890a; PD4006a; PD24190a; PD24325a |
| B4 | PD4833a; PD4847a; PD5948a; PD6406a; PD6728b; PD7066a; PD8611a; PD9571a; |
| | PD9604a; PD9702a; PD18020a; PD8619a; PD9576a; PD4841a; PD23574a; PD23578a; |
| | PD24325a; PD24337a |
| B5 | PD4315a; PD5956a; PD9756a; PD11750a; PD6727b; PD7248a; PD8652a2; PD9702a; |
| | PD13165a; PD7304a; PD24303a; PD24322a; PD24337a |
| B6 | PD11379a; PD4255a; PD4875a; PD8982a; PD9702a; PD22365a; PD24208a; PD24337a |
| B7 | PD5956a; PD11818a; PD4956a; PD5932a; PD5934a; PD5935a; PD5945a; PD6415a; |
| | PD6722a; PD6727b; PD6731a2; PD7067a; PD8611a; PD8652a2; PD9571a; PD9702a; |
| | PD13297a; PD11349a; PD18050a; PD18189a; PD23559a; PD24197a; PD24201a; |
| | PD24216a; PD24217a; PD24325a; PD24337a |
| B8 | PD3989a; PD4953a; PD8612a; PD9464a; PD11336a; PD11379a; PD13771a; PD14453a; |
| | PD6406a; PD7066a; PD7316a; PD8652a2; PD8982a; PD9571a; PD9575a; PD9592a; |
| | PD9595a; PD9702a; PD13297a; PD13311a; PD11465a; PD4826a; PD4841a; PD4005a; |
| | PD4248a; PD22355a; PD23574a; PD23577a; PD23578a; PD24325a; PD24336a; |
| | PD24337a |
| B9 | PD4952a; PD6043a; PD8977a; PD11368a; PD11379a; PD18251a; PD4956a; PD5932a; |
| | PD6728b; PD7066a; PD7316a; PD8660a2; PD8982a; PD9571a; PD9604a; PD9696a; |
| | PD9702a; PD10011a; PD18024a; PD18037a; PD11345a; PD18045a; PD18048a; |
| | PD4841a; PD4198a; PD24208a; PD24209a; PD24314a |
| B10 | PD11742a; PD5935a; PD5948a; PD6409a; PD7066a; PD7316a; PD9571a; PD9584a; |
| | PD9702a; PD10011a; PD18024a; PD23559a; PD23563a; PD23566a; PD24325a |
| B11 | PD6043a; PD8980a; PD11379a; PD4955a; PD6047a; PD8965a; PD9584a; PD9595a; |
| | PD9702a; PD10011a; PD13428a; PD13165a; PD18048a; PD4826a; PD4006a; PD22364a; |
| | PD23559a |
| B12 | PD7243a; PD13422a; PD5935a; PD6727b; PD6728b; PD7316a; PD8660a2; PD8830a; |
| | PD9571a; PD9584a; PD9595a; PD7205a; PD4841a; PD4248a; PD24337a |
| B13 | PD5951a; PD8609a; PD4980a; PD7066a; PD8660a2; PD18020a; PD23559a; PD24322a |
| B14 | PD4604a; PD4959a; PD5956a; PD8610a; PD9756a; PD11743a; PD11818a; PD13757a; |
| | PD14437a; PD18251 a; PD8660a2; PD4841 a; PD24216a |
| B15 | PD6422a; PD14465a; PD4845a; PD5950a; PD7066a; PD7316a; PD8982a; PD13622a; |
| | PD6684a; PD13608a; PD9576a; PD4841a; PD11751a; PD24337a |
| B16 | PD4956a; PD6732b; PD8652a2; PD9604a; PD9702a; PD4841a; PD4006a; PD4109a; |
| | PD23566a |
| B17 | PD5956a; PD14453a; PD5934a; PD6728b; PD7428a; PD8660a2; PD9576a; PD4841a; |
| | PD22355a; PD24202a; PD24308a; PD24325a |
| B18 | PD4613a; PD11336a; PD13752a; PD14437a; PD4874a; PD4956a; PD4980a; PD6415a; |
| | PD6732b; PD7066a; PD7211a; PD9702a; PD10011a; PD7205a; PD9576a; PD7249a; |
| | PD4841a; PD4006a; PD24208a; PD24325a |
| B19 | PD4970a; PD8612a; PD9589a; PD11742a; PD13312a; PD13764a; PD4252a; PD4847a; |
| | PD4956a; PD6406a; PD6733b; PD7066a; PD8982a; PD9571a; PD9592a; PD10014a; |
| | PD13165a; PD18048a; PD6404a; PD4841a; PD11751a; PD4006a; PD4086a; |
| | PD4109a; PD22358a; PD23559a; PD23566a; PD23574a; PD24208a; PD24215a; PD24325a |
| B20 | PD4607a; PD4953a; PD4255a; PD4956a; PD5932a; PD5934a; PD5935a; PD7426a; |
| | PD10011a; PD10014a; PD11748a; PD18048a; PD4841a; PD3890a; PD4006a; |
| | PD4103a; PD23578a; PD24195a; PD24325a |
| B21 | PD11368a; PD4255a; PD7211a; PD8982a; PD8984a; PD9702a; PD10010a; PD13165a; |
| | PD23566a |
| B22 | PD5935a; PD6409a; PD9604a; PD9702a; PD18048a; PD4841a; PD4109a; PD24216a |
| B23 | PD4872a; PD4953a; PD5956a; PD11336a; PD11365a; PD13312a; PD13625a; PD14437a; |
| | PD14453a; PD18251a; PD7066a; PD9702a; PD13165a; PD24216a |
| B24 | PD7215a; PD8978a; PD13312a; PD4255a; PD5935a; PD6406a; PD8660a2; PD10014a; |
| | PD11327a; PD11755a; PD18024a; PD18045a; PD18048a; PD6404a; PD8998a; PD4841a; |
| | PD3905a; PD24325a |
| B25 | PD4953a; PD5956a; PD11343a; PD6728b; PD6732b; PD7066a; PD8611a; PD9579a; |
| | PD9592a; PD9604a; PD11348a; PD22364a; PD23577a |
| B26 | PD6720a; PD7206a; PD8978a; PD9193a; PD9605a; PD11398a; PD14453a; PD7066a; |
| | PD11464a; PD13164a; PD7304a; PD24195a; PD24217a |
| B27 | PD5956a; PD11741a; PD14457a; PD4252a; PD5934a; PD5935a; PD6410a; PD7426a; |
| | PD8611a; PD9696a; PD9702a; PD10014a; PD11345a; PD13166a; PD4841a; PD4005a; |
| | PD23577a; PD24186a; PD24194a; PD24325a; PD24337a |
| B28 | PD8612a; PD4252a; PD4956a; PD5944a; PD6728b; PD7248a; PD7316a; PD8982a; |
| | PD9571a; PD10011a; PD4006a; PD24325a |
| B29 | PD4255a; PD4956a; PD6728b; PD7066a; PD9702a; PD10011a; PD8619a; PD24220a |
| B30 | PD14435a; PD5935a; PD5944a; PD7248a; PD7426a; PD8982a; PD9571a; PD18048a; |
| | PD24303a |
| B31 | PD11337a; PD14457a; PD4847a; PD4956a; PD4980a; PD5934a; PD6727b; PD8660a2; |
| | PD8982a; PD9571a; PD9702a; PD4962a; PD4841a; PD4199a; PD24191a; PD24201a; |
| | PD24207a |
| B32 | PD9467a; PD13312a; PD6732b; PD8660a2; PD9702a; PD18048a; PD4841a; PD4192a; |
| | PD4199a; PD23560a; PD24308a |
| B33 | PD7243a; PD9752a; PD9754a; PD11368a; PD11743a; PD11765a; PD14437a; PD18251a; |
| | PD18264a; PD4833a; PD4956a; PD5942a; PD6732b; PD7066a; PD7426a; PD8964a; |
| | PD8982a; PD8984a; PD9592a; PD9604a; PD9696a; PD9702a; PD10011a; PD18020a; |
| | PD13165a; PD18048a; PD4841a; PD22363a; PD23574a; PD23578a; PD24208a |

TABLE 2
Table headers:

| | |
|---|---|
| hotspot.id | ID of hotspot |
| type | PCF-based analysis |
| Chr | chromosome |
| start.bp | start coordinate (GRCh37) |
| end.bp | end coordinate (GRCh37) |
| length.bp | size of hotspot |
| number.bps | number of rearrangement breakpoints within a hotspot |
| number.bps.clustered | number of breakpoints of clustered rearrangements in the hotspot (will be 0 for |
| | dispersed rearrangements and number.bps of clustered rearrangements) |
| avgDist.bp | average distance between breakpoints in the hotspot, log10 bp |
| no.samples | number of samples with rearrangements in the hotspot |
| ER.percent | percentage of ER and/or PR positive samples |
| TN.percent | percentage of triple negative samples |
| HER2.percent | percentage of HER2 positive samples |
| segment.density | number.bps/length.bp |
| Factor | segment.density/genome wide density of rearrangements |
| d.bg | expected density of breakpoints according to the background model |
| d.obs.exp | segment.density/d.bg |
| fragileSites | fragile sites |
| transposons | IDs of L1 transposon sites coinciding with the hotspot. |
| genes | list of genes coinciding with the hotspot |
| targ.genes | gene hit most frequently by rearrangements (compared to rest of hotspot, |
| | binomial test, Poisson distribution). |
| targ.genes.2 | gene hit most frequently by rearrangements (compared to flanking sequence |
| | of gene, window size 10 kb, binomial test, Poisson distribution). |
| censusGenes | list of cancer census genes within the hotspot (downloaded from COSMIC XX) |
| targ.census.genes | intersection of targ.genes and censusGenes |
| breastGenes | list of breast cancer genes |
| figure.label | included in figure as label |
| amplified.dom | list of genes within 5 mb of the hotspot of clustered rearrangements, classified |
| | as dominant in the census, sorted by frequency across the samples |
| BreastSNPs | breast cancer susceptibility SNPs that overlap with the hotspot |
| superenhancers | super-enhancers that overlap with the hotspot |

| hotspot no. | hotspot.id | type | chr | start.bp | end.bp | length.bp |
|---|---|---|---|---|---|---|
| 1 | peak_RS3_chr13_48.9mb | RS3 | 13 | 48,898,738 | 49,035,729 | 136,991 |
| 2 | peak_RS3_chr7_92mb | RS3 | 7 | 92,044,943 | 92,358,020 | 313,077 |
| 3 | peak_RS3_chr10_89.7mb | RS3 | 10 | 89,678,926 | 89,722,976 | 44,050 |
| 4 | peakRS3_chr11_64.7mb | RS3 | 11 | 64,712,254 | 65,359,025 | 646,771 |

| hotspot no. | number.bps | number.bps.clustered | avgDist.bp | no.samples |
|---|---|---|---|---|
| 1 | 25 | 0 | 3.41134996 | 14 |
| 2 | 29 | 0 | 3.69193267 | 12 |
| 3 | 29 | 0 | 2.93501918 | 15 |
| 4 | 40 | 0 | 3.79402226 | 17 |

| hotspot no. | ER.percent | TN.percent | HER2.percent | segment.density | factor |
|---|---|---|---|---|---|
| 1 | 0 | 93 | 7 | 0.00018249 | 19.1 |
| 2 | 0 | 100 | 0 | 9.26E−05 | 9.7 |
| 3 | 0 | 100 | 0 | 0.00065834 | 68.7 |
| 4 | 6 | 88 | 6 | 6.18E−05 | 6.5 |

| hotspot no. | d.bg | d.obs.exp | fragileSites |
|---|---|---|---|
| 1 | 8.58E−06 | 21.3 | |
| 2 | 1.19E−05 | 7.8 | FRA7E |
| 3 | 9.40E−06 | 70.0 | FRA10A |
| 4 | 1.26E−05 | 4.9 | FRA11H |

| hotspot no. | Genes |
|---|---|
| 1 | RB1; LPAR6 |
| 2 | GATAD1; ERVW-1; PEX1; RBM48; FAM133B; CDK6 |
| 3 | PTEN |
| 4 | C11orf85; BATF2; ARL2; SNX15; SAC3D1; NAALADL1; CDCA5; ZFPL1; |
| | VPS51; TM7SF2; ZNHIT2; FAU; SYVN1; MRPL49; SPDYC; CAPN1; POLA2; |
| | CDC42EP2; DPF2; TIGD3; SLC25A45; FRMD8; SCYL1; LTBP3; SSSCA1; |
| | FAM89B; EHBP1L1 |

| hotspot no. | targ.genes | censusGenes | targ.census.genes | breast Genes | breastSNPs | superenhancers |
|---|---|---|---|---|---|---|
| 1 | RB1; LPAR6 | RB1 | RB1 | RB1 | | |
| 2 | GATAD1; | CDK6 | CDK6 | | | |
| | CDK6 | | | | | |
| 3 | PTEN | PTEN | PTEN | PTEN | | |
| 4 | | | | | | SENH-NEAT1- |
| | | | | | | chr11: 65184888; |
| | | | | | | SENH-MALAT1- |
| | | | | | | chr11: 65238917; |
| | | | | | | SENH-SSSCA1-AS1- |
| | | | | | | chr11: 65323331 |

| hotspot no. | Samples |
|---|---|
| 1 | PD5930a; PD5945a; PD7250a; PD8611a; PD8621a; PD9064a; PD9702a; PD11326a; |
| | PD13296a; PD6684a; PD3905a; PD4005a; PD22366a; PD23566a |
| 2 | PD5935a; PD5945a; PD7211a; PD7248a; PD7426a; PD8652a2; PD9585a; PD9595a; |
| | PD18024a; PD22355a; PD23578a; PD24306a |
| 3 | PD5934a; PD5948a; PD6406a; PD6413a; PD7211a; PD7248a; PD7321a; PD8611a; |
| | PD8621a; PD9585a; PD9702a; PD11755a; PD24202a; PD24303a; PD24306a |
| 4 | PD11742a; PD7211a; PD7316a; PD7321a; PD7426a; PD7428a; PD8611a; PD8652a2; |
| | PD9064a; PD9585a; PD11748a; PD18020a; PD8619a; PD9576a; PD4006a; |
| | PD23577a; PD24197a |

TABLE 3

| | |
|---|---|
| or | odds ratio - enrichment of genomic features in the hotspots |
| | compared to rest of tandem duplicated genome |
| rate | number of elements in the hotspots per basepair |
| rate.upper | upper confidence interval of the element density |
| or.lower | lower confidence interval |
| pvalue | p-value for element enrichment in the hotspots, Poisson test |

| feature | or | rate | rate.upper | or.lower | pvalue |
|---|---|---|---|---|---|
| breast cancer | 4.3 | 1.56E−07 | 2.97E−07 | 7.16E−08 | 3.41E−04 |
| susceptibility | | | | | |
| SNPs | | | | | |
| breast | 3.5 | 1.03E−06 | 1.32E−06 | 7.81E−07 | 6.96E−16 |
| superenhancers | | | | | |
| non-breast | 1.6 | 9.39E−07 | 1.22E−06 | 7.05E−07 | 6.38E−04 |
| superenhancers | | | | | |
| oncogenes | 1.4 | 1.91E−07 | 3.42E−07 | 9.55E−08 | 1.48E−01 |
| promoters | 1.3 | 1.02E−05 | 1.10E−05 | 9.35E−06 | 4.73E−10 |
| enhancers | 1.0 | 5.23E−05 | 5.42E−05 | 5.04E−05 | 1.23E−01 |
| broad fragile | 0.9 | 2.79E−01 | | * | |
| sites | | | | | |
| narrow fragile | 1.3 | 4.07E−02 | | ** | |
| sites | | | | | |

* not tested because of OR
** the statistical test is not suitable for such large elements

TABLE 4

| | | | | | genes |
|---|---|---|---|---|---|
| | | | oncogenes | other | outside |
| coeffi- | | | in RS1 | genes in | of |
| cient | interpretation | c-MYC | hotspots | hotspots | hotspots |
| t | any tandem | 0.99 | | | |
| | duplication | (s.e. 0.28, | | | |
| | in RS1 | p′ = | | | |
| | hotspot | 4.4E−4 ) | | | |
| | (any of the | | | | |
| | 4 below) | | | | |
| dg | tandem | | 0.58 | 0.45 | 0.33 |
| | duplication | | (s.e. 0.17, | (s.e. 0.0, | (s.e. 0.05) |
| | of gene body | | p′ = | p′ = | |
| | | | 6.3E−4) | 1.8E−44) | |
| ds | tandem | | 0.30 | 0.16 | 0.09 |
| | duplication | | (s.e 0.20, | (s.e. 0.04, | (s.e. 0.07) |
| | of super- | | p′ = 0.13) | p′ = | |
| | enhancer or | | | 1.8E−4) | |
| | SNP within | | | | |
| | 1 Mb of | | | | |
| | gene | | | | |
| do | other tandem | | −0.02 | 0.11 | 0.02 |
| | duplication | | (s.e 0.18) | (s.e. 0.03) | (s.e. 0.04) |
| | with 1 Mb | | | | |
| | of gene | | | | |
| dt | tandem | | not | −0.37 | −0.32 |
| | duplication | | frequent | (s.e. 0.34) | (s.e. 0.39) |
| | transecting | | enough | | |
| | the gene | | | | |
| c | background | 0.53 | 0.41 | 0.33 | 0.33 |
| | copy-number | (s.e. 0.09) | (s.e. 0.03) | (s.e. 0.03) | (s.e. 0.01) |
| | of gene | | | | |
| | region | | | | |
| | (ASCAT) | | | | |
| r.HER2 | adjustment | −0.30 | random | random | random |
| | of intercept | (s.e. 0.42) | coefficient | coefficient | coefficient |
| | for HER2+ | | | | |
| | samples | | | | |
| r.TN | adjustment | 0.31 | random | random | random |
| | of intercept | (s.e. 0.14) | coefficient | coefficient | coefficient |
| | for triple | | | | |
| | negative | | | | |
| | samples | | | | |
| intercept | regression | 2.27 | 3.06 | 1.92 | 1.84 |
| | intercept | (s.e. 0.20) | (s.e. 0.34) | (s.e. 0.06) | (s.e. 0.07) |

TABLE 5

| | |
|---|---|
| hotspot.id | ID of hotspot |
| chr | chromosome |
| start.bp | start coordinate (GRCh37) |
| end.bp | end coordinate (GRCh37) |
| length.bp | size of hotspot |
| number.bps | number of rearrangement breakpoints |
| | within a hotspot |
| number.bps.clustered | number of breakpoints of clustered |
| | rearrangements in the hotspot (will |
| | be 0 for dispersed rearrangements |
| | and number.bps of clustered |
| | rearrangements) |
| avgDist.bp | average distance between breakpoints |
| | in the hotspot, log10 bp |
| no.samples | number of samples with rearrangements |
| | in the hotspot |
| d.seg | number.bps/length.bp |
| rate.factor | segment.density/genome wide density |
| | of rearrangements |
| d.bg | expected density of breakpoints |
| | according to the background model |
| d.obs.exp | segment.density/d.bg |

| hotspot no. | hotspot.id | chr | start.bp | end.bp | length.bp |
|---|---|---|---|---|---|
| OV1 | RS1_OV_chr1_150.3Mb | 1 | 150301991 | 156115204 | 5813213 |
| OV2 | RS1_OV_chr10_79.1Mb | 10 | 79065365 | 80026035 | 960670 |
| OV3 | RS1_OV_chr15_71.3Mb | 15 | 71261445 | 72609337 | 1347892 |
| OV4 | RS1_OV_chr2_27.8Mb | 2 | 27825929 | 29225596 | 1399667 |
| OV5 | RS1_OV_chr20_30Mb | 20 | 29959979 | 35657166 | 5697187 |
| OV6 | RS1_OV_chr3_48.6Mb | 3 | 48622102 | 50603571 | 1981469 |
| OV7 | RS1_OV_chr9_33.1Mb | 9 | 33092553 | 36084648 | 2992095 |

| hotspot no. | number.bps | number.bps.clustered | avgDist.bp | no.samples |
|---|---|---|---|---|
| OV1 | 58 | 0 | 4.56684132 | 12 |
| OV2 | 18 | 0 | 4.52464452 | 9 |
| OV3 | 21 | 0 | 4.64509603 | 10 |
| OV4 | 20 | 0 | 4.51984068 | 9 |
| OV5 | 66 | 0 | 4.60248088 | 18 |
| OV6 | 29 | 0 | 4.5076117 | 14 |
| OV7 | 22 | 0 | 4.7446553 | 10 |

| hotspot no. | d.seg | rate.factor | d.bg | d.obs.exp |
|---|---|---|---|---|
| OV1 | 9.98E−06 | 4.74611399 | 4.60E−06 | 2.17056103 |
| OV2 | 1.87E−05 | 8.91301595 | 2.39E−06 | 7.82358965 |
| OV3 | 1.56E−05 | 7.41123537 | 2.34E−06 | 6.66163837 |
| OV4 | 1.43E−05 | 6.79722552 | 3.18E−06 | 4.4878306 |
| OV5 | 1.16E−05 | 5.51073933 | 4.32E−06 | 2.68416676 |
| OV6 | 1.46E−05 | 6.96204976 | 3.35E−06 | 4.36846188 |
| OV7 | 7.35E−06 | 3.49762875 | 2.75E−06 | 2.67032152 |

### REFERENCES

1. Nik-Zainal, S. A compendium of 560 breast cancer genomes. Nature (2016a).
2. Huang, F. W. et al. Highly recurrent TERT promoter mutations in human melanoma. Science 339, 957-9 (2013).
3. Vinagre, J. et al. Frequency of TERT promoter mutations in human cancers. Nat Commun 4, 2185 (2013).
4. Puente, X. S. et al. Non-coding recurrent mutations in chronic lymphocytic leukaemia. Nature 526, 519-24 (2015).
5. Alexandrov, L. B. et al. Signatures of mutational processes in human cancer. Nature 500, 415-21 (2013).
6. Mehta, A. & Haber, J. E. Sources of DNA double-strand breaks and models of recombinational DNA repair. Cold Spring Harb Perspect Biol 6, a016428 (2014).
7. Ceccaldi, R., Rondinelli, B. & D'Andrea, A. D. Repair Pathway Choices and Consequences at the Double-Strand Break. Trends Cell Biol 26, 52-64 (2016).
8. al, M. e. The topography of mutational processes in 560 breast cancer genomes. Nature Communications (2016).
9. Helleday, T., Eshtad, S. & Nik-Zainal, S. Mechanisms underlying mutational signatures in human cancers. Nat Rev Genet 15, 585-98 (2014).
10. Waddell, N. et al. Whole genomes redefine the mutational landscape of pancreatic cancer. Nature 518, 495-501 (2015).
11. Patch, A. M. et al. Whole-genome characterization of chemoresistant ovarian cancer. Nature 521, 489-94 (2015).
12. Menghi, F. et al. The tandem duplicator phenotype as a distinct genomic configuration in cancer. Proc Natl Acad Sci USA 113, E2373-82 (2016).
13. McBride, D. J. et al. Tandem duplication of chromosomal segments is common in ovarian and breast cancer genomes. J Pathol 227, 446-55 (2012).
14. Stephens, P. J. et al. Complex landscapes of somatic rearrangement in human breast cancer genomes. Nature 462, 1005-10 (2009).
15. Nik-Zainal, S. et al. Mutational processes molding the genomes of 21 breast cancers. Cell 149, 979-93 (2012).
16. Nilsson, B., Johansson, M., Heyden, A., Nelander, S. & Fioretos, T. An improved method for detecting and delineating genomic regions with altered gene expression in cancer. Genome Biol 9, R13 (2008).
17. Nilsen, G. et al. Copynumber: Efficient algorithms for single- and multi-track copy number segmentation. BMC Genomics 13, 591 (2012).
18. Garcia-Closas, M. et al. Genome-wide association studies identify four ER negative-specific breast cancer risk loci. Nat Genet 45, 392-8, 398e1-2 (2013).
19. Easton, D. F. et al. Genome-wide association study identifies novel breast cancer susceptibility loci. Nature 447, 1087-93 (2007).
20. Li, S. et al. Endocrine-therapy-resistant ESR1 variants revealed by genomic characterization of breast-cancer-derived xenografts. Cell Rep 4, 1116-30 (2013).
21. Robinson, D. R. et al. Activating ESR1 mutations in hormone-resistant metastatic breast cancer. Nat Genet 45, 1446-51 (2013).
22. Soucek, L. et al. Modelling Myc inhibition as a cancer therapy. Nature 455, 679-83 (2008).
23. Shi, J. et al. Role of SWI/SNF in acute leukemia maintenance and enhancer-mediated Myc regulation. Genes Dev 27, 2648-62 (2013).
24. Zhang, X. et al. Identification of focally amplified lineage-specific super-enhancers in human epithelial cancers. Nat Genet 48, 176-82 (2016).
25. Costantino, L. et al. Break-induced replication repair of damaged forks induces genomic duplications in human cells. Science 343, 88-91 (2014).
26. Willis, N. A., Rass, E. & Scully, R. Deciphering the Code of the Cancer Genome: Mechanisms of Chromosome Rearrangement. Trends Cancer 1, 217-230 (2015).
27. Saini, N. et al. Migrating bubble during break-induced replication drives conservative DNA synthesis. Nature 502, 389-92 (2013).
28. Sloan, C. A. et al. ENCODE data at the ENCODE portal. Nucleic Acids Res 44, D726-32 (2016).
29. Castro-Giner, F., Ratcliffe, P. & Tomlinson, I. The mini-driver model of polygenic cancer evolution. Nat Rev Cancer 15, 680-5 (2015).
30. Roy, A. et al. Recurrent internal tandem duplications of BCOR in clear cell sarcoma of the kidney. Nat Commun 6, 8891 (2015).
31. Ahmed, S., Thomas, G., Ghoussaini, M., Healey, C. S., Humphreys, M. K., Platte, R., Morrison, J., Maranian, M., Pooley, K. A., Luben, R., et al. (2009). Newly discovered breast cancer susceptibility loci on 3p24 and 17q23.2. Nature genetics 41, 585-590.
32. Bignell, G. R., Greenman, C. D., Davies, H., Butler, A. P., Edkins, S., Andrews, J. M., Buck, G., Chen, L., Beare, D., Latimer, C., et al. (2010). Signatures of mutation and selection in the cancer genome. Nature 463, 893-898.
33. Cox, A., Dunning, A. M., Garcia-Closas, M., Balasubramanian, S., Reed, M. W., Pooley, K. A., Scollen, S., Baynes, C., Ponder, B. A., Chanock, S., et al. (2007). A common coding variant in CASP8 is associated with breast cancer risk. Nature genetics 39, 352-358.
34. Easton, D. F., Deffenbaugh, A. M., Pruss, D., Frye, C., Wenstrup, R. J., Allen-Brady, K., Tavtigian, S. V., Monteiro, A. N., Iversen, E. S., Couch, F. J., et al. (2007). A systematic genetic assessment of 1,433 sequence variants of unknown clinical significance in the BRCA1 and BRCA2 breast cancer-predisposition genes. American journal of human genetics 81, 873-883.
35. Michailidou, K., Beesley, J., Lindstrom, S., Canisius, S., Dennis, J., Lush, M. J., Maranian, M. J., Bolla, M. K., Wang, Q., Shah, M., et al. (2015). Genome-wide association analysis of more than 120,000 individuals identifies 15 new susceptibility loci for breast cancer. Nature genetics 47, 373-380.
36. Nik-Zainal, S. (2016b). Landscape of somatic mutations in 560 whole-genome sequenced breast cancers.
37. Siddiq, A., Couch, F. J., Chen, G. K., Lindstrom, S., Eccles, D., Millikan, R. C., Michailidou, K., Stram, D. O., Beckmann, L., Rhie, S. K., et al. (2012). A meta-analysis of genome-wide association studies of breast cancer identifies two novel susceptibility loci at 6q14 and 20q11. Human molecular genetics 21, 5373-5384.
38. Stacey, S. N., Manolescu, A., Sulem, P., Thorlacius, S., Gudjonsson, S. A., Jonsson, G. F., Jakobsdottir, M., Bergthorsson, J. T., Gudmundsson, J., Aben, K. K., et al. (2008). Common variants on chromosome 5p12 confer susceptibility to estrogen receptor-positive breast cancer. Nature genetics 40, 703-706.
39. Thomas, G., Jacobs, K. B., Kraft, P., Yeager, M., Wacholder, S., Cox, D. G., Hankinson, S. E., Hutchinson, A., Wang, Z., Yu, K., et al. (2009). A multistage genome-wide association study in breast cancer identifies two new risk alleles at 1p11.2 and 14q24.1 (RAD51L1). Nature genetics 41, 579-584.
40. Turnbull, C., Ahmed, S., Morrison, J., Pernet, D., Renwick, A., Maranian, M., Seal, S., Ghoussaini, M., Hines, S., Healey, C. S., et al. (2010). Genome-wide association study identifies five new breast cancer susceptibility loci. Nature genetics 42, 504-507.
41. Wei, Y., Zhang, S., Shang, S., Zhang, B., Li, S., Wang, X., Wang, F., Su, J., Wu, Q., Liu, H., et al. (2016). SEA: a super-enhancer archive. Nucleic acids research 44, D172-179.
42. Zerbino, D. R., and Birney, E. (2008). Velvet: algorithms for de novo short read assembly using de Bruijn graphs. Genome research 18, 821-829.
43. Zerbino, D. R., Wilder, S. P., Johnson, N., Juettemann, T., and Flicek, P. R. (2015). The ensembl regulatory build. Genome biology 16, 56.

1. A method of classifying a breast cancer, comprising
testing DNA from said breast cancer for the presence of chromosomal rearrangement within 10 or more of the rearrangement hotspots defined in Table 1; and
classifying said breast cancer as deficient in homologous recombination repair (HR-deficient) if rearrangement is identified in at least one of said rearrangement hotspots.

2. A method according to claim 1 comprising testing for the presence of chromosomal rearrangement within 15 or more, within 20 or more, within 25 or more, within 26 or more, 27 or more, 28 or more, 29 or more, 30 or more, 31 or more, 32 or more, or all 33 of the hotspots defined in Table 1.

3. A method according to claim 1 or claim 2 comprising classifying the cancer as HR-deficient if rearrangement is identified in each of at least 3 hotspots, at least 4 hotspots, at least 5 hotspots or at least 6 hotspots.

4. A method of determining a therapy for a subject having breast cancer, the method comprising
testing DNA from said breast cancer for the presence of chromosomal rearrangement within 10 or more of the rearrangement hotspots defined in Table 1; and
selecting the subject for treatment with an agent for treatment of HR-deficient cancers if rearrangement is identified in at least one of said rearrangement hotspots.

5. A method according to claim 4 comprising testing for the presence of chromosomal rearrangement within 15 or more, within 20 or more, within 25 or more, within 26 or more, 27 or more, 28 or more, 29 or more, 30 or more, 31 or more, 32 or more, or all 33 of the hotspots defined in Table 1.

6. A method according to claim 4 or claim 5 comprising selecting the subject for treatment if rearrangement is identified in each of at least 3 hotspots, at least 4 hotspots, at least 5 hotspots or at least 6 hotspots.

7. A method according to any one of preceding claims comprising determining a data set for each of the tested hotspots from the cancer DNA and comparing each data set from the cancer DNA with a corresponding reference data set derived from a corresponding reference sequence to identify chromosomal rearrangement in the cancer DNA.

8. A method according to claim 7 wherein the reference sequence is derived from healthy tissue from the same subject.

9. A method according to any one of the preceding claims wherein the DNA from the cancer is genomic DNA or a fraction thereof enriched for sequences within the hotspots to be tested.

10. A method according to claim 9 wherein the genomic DNA is obtained from peripheral blood or from a biopsy.

11. A method according to any one of the preceding claims, wherein detecting chromosomal rearrangement comprises determining the whole or partial sequence of a hotspot or a portion thereof, determining copy number of a particular sequence within the hotspot, or determining the distance between two loci within the hotspot.

12. A method according to any one of the preceding claims, wherein said detection is performed by a method comprising sequencing or hybridisation.

13. A method according to claim 12 wherein said sequencing is performed by paired end sequencing, mate-pair sequencing, targeted sequencing, single molecule real-time sequencing, ion semiconductor (Ion Torrent) sequencing, sequencing by synthesis, sequencing by ligation (SOLiD), nano-pore sequencing or pyrosequencing.

14. A method according to claim 12 wherein said hybridisation comprises array comparative genomic hybridisation (array CGH).

15. A method according to any one of the preceding claims wherein the rearrangement is a tandem duplication.

16. A method of treatment of breast cancer, in a subject
(i) having a breast cancer which has been determined to be HR-deficient by a method according to any one of claims 1 to 3, or any one of claims 7 to 14 as dependent from any one of claims 1 to 3; or
(ii) selected by a method according to any one of claims 4 to 6, or any one of claims 7 to 14 as dependent from any one of claims 4 to 6;
the method comprising administering an agent for treatment of HR-deficient cancers to the subject.

17. An agent for treatment of HR-deficient cancers, for use in the treatment of breast cancer in a subject
(i) having a breast cancer which has been determined to be HR-deficient by a method according to any one of claims 1 to 3, or any one of claims 7 to 14 as dependent from any one of claims 1 to 3; or
(ii) selected by a method according to any one of claims 4 to 6, or any one of claims 7 to 14 as dependent from any one of claims 4 to 6.

18. A method according to claim 16, or an agent for use according to claim 17, wherein the agent is a PARP inhibitor, platinum-based anti-neoplastic agent, anthracycline, topoisomerase I inhibitor or Wee1 inhibitor.

19. A method of classifying an ovarian cancer, comprising
testing DNA from said ovarian cancer for the presence of chromosomal rearrangement within 2 or more of the rearrangement hotspots defined in Table 5; and
classifying said ovarian cancer as deficient in homologous recombination repair (HR-deficient) if rearrangement is identified in at least one of said rearrangement hotspots.

20. A method according to claim 19 comprising testing for the presence of chromosomal rearrangement within 3 or more, within 4 or more, within 5 or more, within 6 or more, or within all 7 hotspots defined in Table 5.

21. A method according to claim 19 or claim 20 comprising classifying the cancer as HR-deficient if chromosomal rearrangement is identified in each of at least 2 hotspots, at least 3 hotspots, at least 4 hotspots, at least 5 hotspots, at least 6 hotspots, or all 7 hotspots.

22. A method of determining a therapy for a subject having an ovarian cancer, the method comprising
testing DNA from said ovarian cancer for the presence of chromosomal rearrangement within 2 or more of the rearrangement hotspots defined in Table 5; and
selecting the subject for treatment with an agent for treatment of HR-deficient cancers if rearrangement is identified in at least one of said rearrangement hotspots.

23. A method according to claim 22 comprising testing for the presence of chromosomal rearrangement within 3 or more, within 4 or more, within 5 or more, within 6 or more, or within all 7 hotspots defined in Table 5.

24. A method according to claim 22 or claim 23 comprising selecting the subject for treatment if chromosomal rearrangement is identified in each of at least 2 hotspots, at least 3 hotspots, at least 4 hotspots, at least 5 hotspots, at least 6 hotspots, or all 7 hotspots.

25. A method according to any one of claims 19 to 24 comprising determining a data set for each of the tested hotspots from the cancer DNA and comparing each data set from the cancer DNA with a corresponding reference data set derived from a corresponding reference sequence to identify chromosomal rearrangement in the cancer DNA.

26. A method according to claim 25 wherein the reference sequence is derived from healthy tissue from the same subject.

27. A method according to any one of claims 19 to 26, wherein the DNA from the cancer is genomic DNA or a fraction thereof enriched for sequences within the hotspot to be tested.

28. A method according to claim 27 wherein the genomic DNA is obtained from peripheral blood or from a biopsy.

29. A method according to any one of claims 19 to 28, wherein detecting chromosomal rearrangement comprises determining the whole or partial sequence of a hotspot or a portion thereof, determining a change in copy number of a particular sequence within the hotspot, or determining the distance between two loci within the hotspot.

30. A method according to any one of claims 19 to 29, wherein said detection is performed by a method comprising sequencing or hybridisation.

31. A method according to claim 30 wherein said sequencing is performed by paired end sequencing, mate-pair sequencing, targeted sequencing, single molecule real-time sequencing, ion semiconductor (Ion Torrent) sequencing, sequencing by synthesis, sequencing by ligation (SOLiD), nano-pore sequencing or pyrosequencing.

32. A method according to claim 30 wherein said hybridisation comprises array comparative genomic hybridisation (array CGH).

33. A method according to any one of claims 19 to 32 wherein the rearrangement is a tandem duplication.

34. A method of treatment of ovarian cancer, in a subject
(i) having ovarian cancer which has been determined to be HR-deficient by a method according to any one of claims 19 to 21, or any one of claims 25 to 33 as dependent from any one of claims 18 to 20; or
(ii) selected by a method according to any one of claims 22 to 24, or any one of claims 25 to 33 as dependent from any one of claims 22 to 24;
the method comprising administering an agent for treatment of HR-deficient cancers to the subject.

35. An agent for treatment of HR-deficient cancers, for use in the treatment of ovarian cancer in a subject
(i) having ovarian cancer which has been determined to be HR-deficient by a method according to any one of claims 19 to 21, or any one of claims 25 to 32 as dependent from any one of claims 19 to 21; or
(ii) selected by a method according to any one of claims 22 to 24, or any one of claims 25 to 33 as dependent from any one of claims 22 to 24.

36. A method according to claim 34, or an agent for use according to claim 35, wherein the agent is a PARP inhibitor, platinum-based anti-neoplastic agent, anthracycline, topoisomerase I inhibitor or Wee1 inhibitor.

37. A method of classifying a breast cancer, comprising
testing DNA from said breast cancer for the presence of chromosomal rearrangement within hotspot B23 (peak_RS1_chr6_151.8mb) defined in Table 1; and
classifying said breast cancer as ER-positive if rearrangement is identified in said hotspot.

38. A method of determining a therapy for a subject having breast cancer, the method comprising
testing DNA from said breast cancer for the presence of chromosomal rearrangement within hotspot B23 (peak_RS1_chr6_151.8mb) defined in Table 1; and
selecting the subject for treatment with an agent for treatment of ER-positive cancers if rearrangement is identified in said hotspot.

39. A method according to claim 37 or claim 38 further comprising testing the copy number of the ESR1 gene.

40. A method according to any one of claims 37 to 39 further comprising testing the ER status of the cancer.

41. A method according to claim 40 comprising testing for expression of ESR1 receptor protein or mRNA.

42. A method according to any one of claims 37 to 41 comprising determining a data set for the hotspot from the cancer DNA and comparing the data set from the cancer DNA with a corresponding reference data set derived from a corresponding reference sequence to identify chromosomal rearrangement in the cancer DNA.

43. A method according to claim 42 wherein the reference sequence is derived from healthy tissue from the same subject.

44. A method according to any one of claims 37 to 43 wherein the DNA from the cancer is genomic DNA or a fraction thereof enriched for sequences within the hotspots to be tested.

45. A method according to claim 44 wherein the genomic DNA is obtained from peripheral blood or from a biopsy.

46. A method according to any one of claims 37 to 45 wherein detecting chromosomal rearrangement comprises determining the whole or partial sequence of the hotspot or a portion thereof, determining copy number of a particular sequence within the hotspot, or determining the distance between two loci within the hotspot.

47. A method according to any one of claims 37 to 46, wherein said detection is performed by a method comprising sequencing or hybridisation.

48. A method according to claim 47 wherein said sequencing is performed by paired end sequencing, mate-pair sequencing, targeted sequencing, single molecule real-time sequencing, ion semiconductor (Ion Torrent) sequencing, sequencing by synthesis, sequencing by ligation (SOLID), nano-pore sequencing or pyrosequencing.

49. A method according to claim 47 wherein said hybridisation comprises array comparative genomic hybridisation (array CGH).

50. A method according to any one of claims 37 to 49 wherein the rearrangement is a tandem duplication.

51. A method of treatment of breast cancer, in a subject
(i) having a breast cancer which has been determined to be ER-positive by a method according to claim 37 or any one of claims 39 to 50 as dependent from claim 37;
(ii) selected by a method according to 38; or any one of claims 39 to 50 as dependent from claim 38;
the method comprising administering an agent for treatment of ER-positive cancers to the subject.

52. An agent for use in the treatment of ER-positive cancers, for use in the treatment of breast cancer in a subject
(i) having a breast cancer which has been determined to be ER-positive by a method according to claim 37 or any one of claims 39 to 50 as dependent from claim 37;
(ii) selected by a method according to 38; or any one of claims 39 to 50 as dependent from claim 38.

53. A method according to claim 51 or an agent for use according to claim 52, wherein the agent is a selective estrogen-receptor response modulator (SERM), an aromatase inhibitor, an estrogen receptor downregulator (ERD), or a luteinizing hormone-releasing hormone agent (LHRH).
